(19) United States
(12) Patent Application Publication
(10) Pub. No.: US 2010/0280759 A1
(43) Pub. Date: Nov. 4, 2010

(54) MASS SPECTROMETER OUTPUT ANALYSIS TOOL FOR IDENTIFICATION OF PROTEINS

(75) Inventors: Oren Kagan, Chestnut Hill, MA (US); James R. Dasch, Needham, MA (US); Aaron Sin, St. Louis, MO (US)

Correspondence Address: WOLF GREENFIELD & SACKS, P.C., 600 ATLANTIC AVENUE, BOSTON, MA, 02210-2206, US

(73) Assignee: Cell Biosciences, San Leandro, CA (US)

(21) Appl. No.: 12/473,005

(22) Filed: May 27, 2009

Related U.S. Application Data
(60) Provisional application No. 61/057,393, filed on May 30, 2008.

(51) Int. Cl. G06F 19/00; G01N 33/68

(52) U.S. Cl. 702/19

(57) ABSTRACT

Methods and apparatus for consolidation and visualization of a dataset output from a mass spectrometer. The number of proteins identified in a mass spectrometry experiment may be systematically reduced by identifying and excluding proteins which may not be necessary to explain the observed peptides in a sample. Peptide counts from excluded proteins may be redistributed to non-excluded proteins to preserve total observed peptide counts. The consolidated dataset may be represented as a data structure having a plurality of cells arranged into rows and columns. Visual parameter values may be assigned to various cells in the data structure to emphasize particular features of the data. Data from multiple samples may be arranged in sets of columns in the data structure, and difference scores may be calculated to determine peptide expression differences between the multiples samples. The difference scores may be displayed using textual and/or graphical representations to facilitate data analysis.

### RELATED APPLICATIONS

This application claims priority under 35 U.S.C. §119(e) to U.S. Provisional Application Ser. No. 61/057,393 entitled “MASS SPECTROMETER OUTPUT ANALYSIS TOOL FOR IDENTIFICATION OF PROTEINS,” filed on May 30, 2008, which is herein incorporated by reference in its entirety.

### TECHNICAL FIELD

The present invention relates to the visualization and analysis of output from a mass spectrometer to facilitate identification of proteins in samples.

### BACKGROUND

Mass spectrometry is an analytical technique that measures the mass-to-charge ratio of charged particles in a sample. A mass spectrometer may be used to determine the composition of a sample by characterizing ions in the sample based on their charge. In the field of proteomics, mass spectrometry has become an important tool in identifying the protein content of complex protein mixtures such as blood or tissue samples. In particular, tandem mass spectrometry (MS/MS) has become a preferred spectrometry method for high-throughput proteomics studies. To analyze protein mixtures using tandem mass spectrometry, proteins in a sample are first proteolytically cleaved into smaller peptide segments using an enzyme such as trypsin, for example. The resultant peptide segments are then fragmented into ions in a mass spectrometer using collision-induced dissociation. The fragmented ions have mass differences corresponding to the residue masses of their respective amino acids. Thus, the tandem mass spectrum contains partial information about the amino acid sequence of the peptides in the sample and this information can be cross-referenced to a database to identify the peptides in a sample based on the detected amino acid sequences. The output from a tandem mass spectrometry experiment is a long list of detected constituent peptides and the possible proteins to which they may belong.

### SUMMARY

While the set of all possible proteins based on detected constituent peptides in a sample is provided in the output of a mass spectrometer, the false positive rate is quite high. That is, many of the proteins listed as possible candidates are not present in the sample, but they merely happen to share peptide sequences with proteins that are present in the sample. Significant overlap in peptide sequences across proteins is not surprising as peptides are relatively short polymers of amino acids that when linked together form the greater than 500,000 proteins in the human body. We have recognized and appreciated that because the number of false positives is large, sorting through the output of a mass spectrometry experiment may be a time consuming and arduous process. Thus, improved methods and apparatus for organizing and visualizing the output of a mass spectrometry experiment are desirable. Such improved methods and apparatus are shown herein.

Some embodiments are directed to methods and apparatus for organizing output from a mass spectrometer, the output comprising a list of a plurality of detected proteins from a sample, each of the plurality of detected proteins having constituent peptides, each constituent peptide having at least one molecular property. The method comprises receiving the output from the mass spectrometer, operating a processor to assign each of the constituent peptides for each detected protein into one of a plurality of bins based at least in part on the at least one molecular property of the peptide, calculating peptide counts indicating a number of said peptides in each bin for each detected protein, displaying the peptide counts as a plurality of cells in a data structure, the display presenting the counts in a table having columns arranged along a first axis and rows arranged along a second axis, the intersection of a column and a row defining a cell, and in each row displaying data pertaining to a specific detected protein and in each column displaying peptide counts for one of said plurality of bins, assigning a first visual parameter value to each column of the data structure and assigning a second visual parameter value to each cell based on its respective peptide count. (Note: rows and columns are defined by the first and second axes, not by their horizontal and vertical orientations).

Some embodiments are directed to computer-implemented methods and apparatus for arranging peptide count information from a mass spectrometer experiment. The method comprises receiving the peptide count information from a mass spectrometer, creating a data structure, the data structure comprising, for each protein for which a peptide count is to be displayed, a plurality of cells, the plurality of cells being arranged along a first axis, and populating the data structure with the peptide count information by assigning to each cell the peptide count obtained over a predefined range of pH.

Some embodiments are directed to methods and apparatus for selecting candidate proteins using mass spectrometry. The method comprises detecting with a mass spectrometer, in a first sample and a second sample, a protein having a respective first number and second number of constituent peptides, each peptide having at least one molecular property, sorting into first bins based on respective one of the at least one molecular properties, the first number of constituent peptides detected in the first sample, sorting into second bins based on respective one of the at least one molecular properties, the second number of constituent peptides detected in the second sample, calculating a difference score for the protein, wherein the difference score represents a measure of difference between a number of peptides in the first bins and a number of peptides in the second bins, and determining that the protein is a candidate protein if the difference score is higher or lower than a predetermined value.

Some embodiments are directed to a computer system for displaying mass spectrometry output. The computer system comprises a computer data structure comprising a plurality of cells, the cells being arranged into at least one first axis for representing at least one protein in the mass spectrometry output and at least one second axis for representing a molecular property and at least one processor programmed to manipulate the computer data structure. The at least one processor comprises a color coding module which codes differences along the at least one second axis, a saturation module which codes magnitudes of values stored in the plurality of cells, and a difference score module which calculates a measure of difference between values stored in the plurality of cells.

Some embodiments are directed to methods and apparatus for consolidating a data set generated by a mass spectrometer, the data set comprising a plurality of candidate proteins having respective constituent peptides. The method comprises receiving the data set from the mass spectrometer, creating a peptide data structure comprising a plurality of first fields for storing information about each of the constituent peptides, searching the peptide data structure for at least two constituent peptides having a nearly-identical molecular sequence, determining, for the at least two constituent peptides having a nearly-identical molecular sequence, a portion of the molecular sequence common to the at least two constituent peptides, and merging the at least two constituent peptides into a single constituent peptide in the peptide data structure having the portion of the molecular sequence.

Some embodiments are directed to methods and apparatus for consolidating a data set generated by a mass spectrometer, the data set comprising a plurality of candidate proteins having respective constituent peptides. The method comprises receiving the data set from the mass spectrometer, defining at least one similarity group comprising at least two candidate proteins, the at least two candidate proteins having at least one common constituent peptide, determining a subset of candidate proteins of the at least two candidate proteins in the at least one similarity group that have at least one exclusive peptide, the at least one exclusive peptide being present in a single candidate protein of the at least two candidate proteins in the at least one similarity group, redistributing constituent peptide counts from the candidate proteins excluded from the subset to candidate proteins included in the subset to form a consolidated data set, and outputting an indication of the consolidated data set.

Some embodiments are directed to computer-implemented methods and apparatus for identifying a subset of proteins from a list of proteins comprising at least one constituent peptide, each protein in the subset comprising at least one exclusive constituent peptide. The method comprises receiving the list of proteins as output from a mass spectrometer, creating a tree-like data structure comprising a plurality of parent nodes, each parent node of the plurality of parent nodes corresponding to a protein in the list of proteins and having at least one child node corresponding to the at least one constituent peptide of the protein corresponding to the parent node, traversing the data structure to identify exclusive parent nodes that have at least one child node not shared by other parent nodes, and outputting the proteins identified by the exclusive parent nodes as the subset of proteins that have at least one exclusive constituent peptide.

Some embodiments are directed to methods and apparatus for processing a list of proteins output from a mass spectrometer, each protein in the list of proteins having constituent peptides. The method comprises receiving the list of proteins from the mass spectrometer, assigning a first protein in the list of proteins to at least one similarity group which includes at least one second protein having a common constituent peptide, classifying each of the first protein and the second protein based at least in part on their respective constituent peptides, producing, for each similarity group, a subset of proteins by eliminating at least some proteins based on their classification, and outputting an indication of the subset of proteins for each similarity group.

Some embodiments are directed to a computer-readable medium encoded with a series of instructions that when executed on a computer perform a method for displaying data output from a mass spectrometer. The computer readable medium comprises a data structure for storing at least the data in a plurality of fields, a plurality of visualization modules linked to the data structure and configured to display portions of the data, wherein at least one property of each of the plurality of visualization modules is user-configurable, and an update module for updating information in at least one of the plurality of visualization modules in response to selection of an object displayed in one of the plurality of visualization modules.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein.

### BRIEF DESCRIPTION OF THE DRAWINGS

The accompanying drawings are not intended to be drawn to scale. In the drawings, each identical or nearly identical component that is illustrated in various figures is represented by a like reference character. For purposes of clarity and the avoidance of obfuscating repetition, not every component may be labeled in every drawing. In the drawings:

FIG. 1 is an example of a mass spectrometer output for use with some embodiments of the invention;

FIG. 2 is a flowchart of a process for detection and consolidation of similar peptides according to some embodiments of the invention;

FIGS. 3A and 3B are respective schematics of a protein data structure and a global peptide list data structure according to some embodiments of the invention;

FIG. 4 is a flowchart of a process for creating an overall coverage map according to some embodiments of the invention;

FIG. 5 is a flowchart of a process for assigning tags to proteins according to some embodiments of the invention;

FIG. 6 is an example of proteins and constituent peptides for use with some embodiments of the invention;

FIGS. 7A and 7B are respective diagrams of a linked list schematic and linked list data structure for use with some embodiments of the invention;

FIG. 8 is a flowchart of a labeling process according to some embodiments of the invention;

FIG. 9 is a flowchart of an exclusion process according to some embodiments of the invention;

FIG. 10 is an overview flowchart of a consolidation process according to some embodiments of the invention;

FIG. 11 is a flowchart of a peptide redistribution process according to some embodiments of the invention;

FIG. 12 is an example of peptide redistribution using the process illustrated in FIG. 11;

FIG. 13 is a flowchart of a consolidation process according to some embodiments of the invention;

FIG. 14 is an example of a data structure for representing peptide counts according to some embodiments of the invention;

FIG. 15 is a schematic representation of a data structure with modifying modules according to some embodiments of the invention;

FIG. 16 is a flowchart of a difference score calculation process according to some embodiments of the invention;

FIG. 17 is an example of a bubble chart for displaying difference score data according to some embodiments of the invention;

FIG. 18 is an example of a bar chart for displaying difference score data according to some embodiments of the invention;

FIG. 19 is an example of a line chart for displaying difference score data according to some embodiments of the invention;

FIG. 20 is an example of a Venn diagram for displaying the proportion of exclusive proteins in at least two samples according to some embodiments of the invention

FIGS. 21A and 21B show a display produced by a portion of a software program for implementing some embodiments of the invention;

FIGS. 22A and 22B show a display produced by a portion of a software program for implementing some embodiments of the invention; and

FIG. 23 is a schematic of a system on which some embodiments of the invention may be employed.

### DETAILED DESCRIPTION

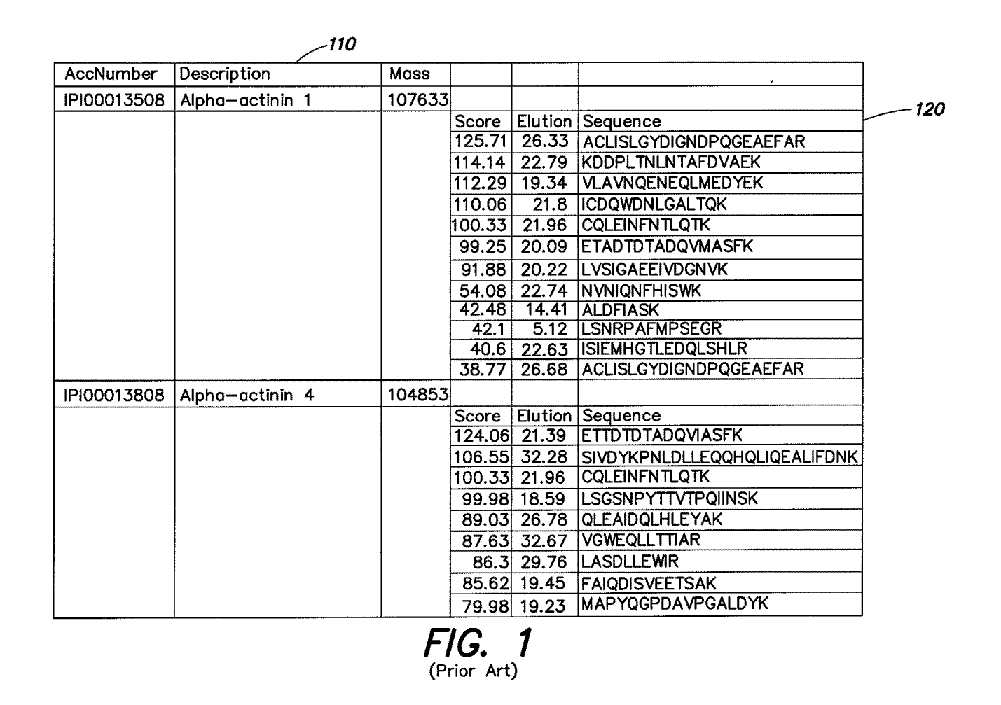

The output of a mass spectrometry experiment may be a structured hierarchical dataset comprising the results from multiple samples. For each sample, a mass spectrum may be generated from which the identity of peptides in the sample may be determined. Software tools such as SEQUEST (available from http://fields.scripps.edu/sequest) or Mascot (from Matrix Science of London, United Kingdom), which shall be referred to herein as “searching” tools or algorithms, may be used to identify the peptides and generate a list of all possible proteins which contain the peptide sequences identified from the mass spectrum. A typical output generated by the searching stage may be a list of proteins and constituent peptides as shown in FIG. 1. Each identified protein 110 may have a list of observed peptides 120 that were used to identify the protein 110, and the protein 110 and/or the peptides 120 may be represented by its molecular sequence.

However, these output lists may be replete with errors introduced as a result of “guessing” during the protein identification process. For example, the peptide information provided to the searching algorithms may not be sufficient to identify uniquely a protein associated with the peptide, resulting in a “best guess” by the searching algorithm. In some instances, the guess may be random, thereby creating logical contradictions in a mass spectroscopy dataset when examined across multiple samples. Additionally, the search databases used to identify peptides and/or proteins may contain duplicate entries, many peptides may be present in more than one protein, and/or the protein may have multiple isoforms.

Some embodiments of the invention are directed to addressing at least some of the aforementioned difficulties in analyzing mass spectrometer output by consolidating long lists of proteins and constituent peptides provided as output from a searching algorithm. We have recognized that peptides are occasionally observed in mass spectrometry in variations, where each instance is slightly different. For example, a peptide may have the same molecular sequence as another peptide, but with an additional amino acid at the C-terminus or the N-terminus. Such minor differences between similar peptides may be considered as sample preparation or biological artifacts, and the similar peptides may be treated as identical.

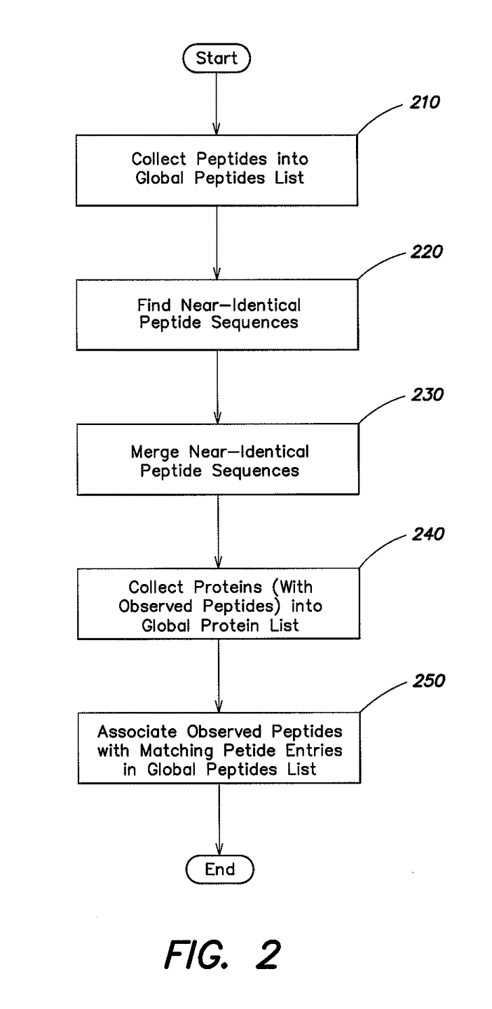

In some embodiments, detection and consolidation of similar peptides may proceed according to a process having a series of steps as illustrated in FIG. 2. In step 210, all of the peptides may be collected into a global peptides list. For example, a data structure representing a list or collection of “global peptides” may be defined (e.g., in a computer-readable data store or memory) and descriptors of all observed peptides in an input dataset with a unique molecular sequence may be used to populate the data structure. Peptides with exactly the same peptide sequence may be detected across multiple samples, and thus in some embodiments, only non-identical observed peptides may be included in the global peptides list.

After creating a global peptides list, the list may be searched in step 220 to find groups of peptides with molecular sequences which are nearly identical. In some embodiments, near-identical peptides may have identical kernel molecular sequences (e.g., C-X-N, where X is a shared string of amino acids), with an additional short sequence of amino acids at the C terminus or N terminus of one of the peptides (e.g., C-X-N and C-X-N-A, where A is a short sequence of amino acids). It should be appreciated that any suitable criteria may be used to determine which peptides in the global peptides list may be considered nearly identical, and embodiments of the invention are not limited in this respect. For example, in one embodiment, a criterion for nearly-identical peptides may be that the additional short sequence of amino acids may be shorter than or equal to two amino acids.

Upon identifying the groups of nearly-identical peptides in the global peptide list, each nearly-identical peptide group may be merged in step 230 into a single peptide entry represented by the group's kernel sequence (i.e., the common substring of amino acids across all near-identical peptides in a group). For example, four peptides may be identified in the global peptide list having the sequences B-C-X-N, C-X-N, C-X-N-A, and C-X-N-E. According to one embodiment, these four peptides may be merged into a single peptide entry having the molecular sequence C-X-N, thereby reducing the peptide level redundancies present in the global peptide list.

Because some of the observed peptides may have been eliminated from the global peptide list in the aforementioned steps, associations between identified proteins with their observed constituent peptides and the remaining peptides in the consolidated global peptide list may be established. In some embodiments, this may be accomplished by collecting proteins observed across all samples into a global protein list, in step 240.

For example, a data structure representing “global proteins” may be defined in a computer-readable data store or memory, and descriptors of all identified proteins and their constituent peptides in the input dataset may be used to populate the data structure.

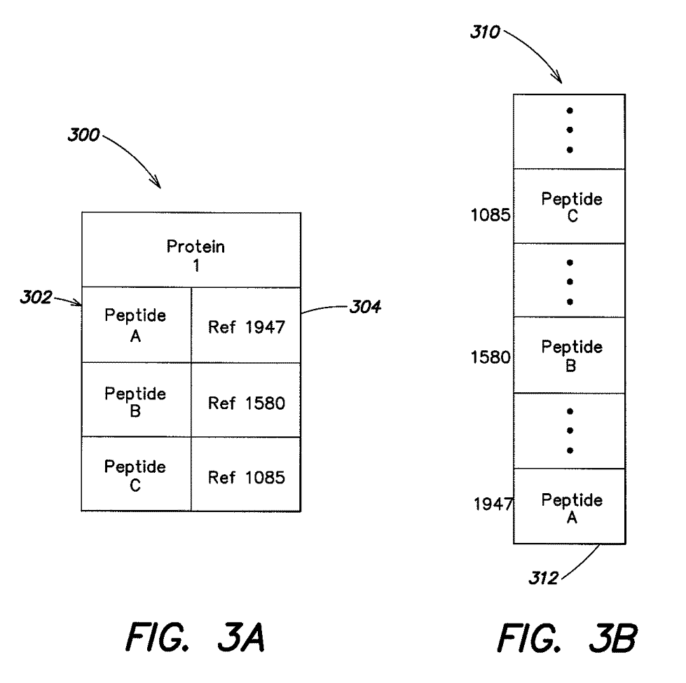

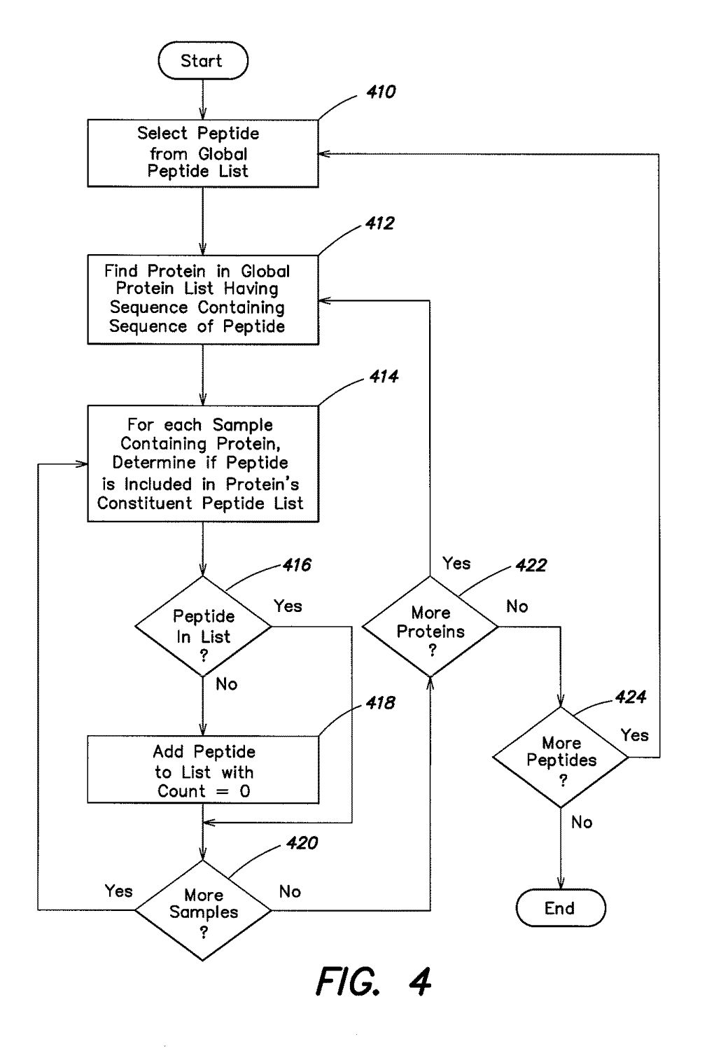

After forming the global protein list, each of the observed peptides for each identified protein in the global protein list may be associated with a matching entry in the consolidated global peptide list, in step 250. In some embodiments, the associations between observed peptides and peptides in the global peptide list may be implemented using a linked-list data structure, as shown in FIGS. 3A and 3B. For example, each protein in the global protein list may be represented by a protein data structure 300 having a plurality of fields for storing information about the protein's constituent peptides. Each observed peptide 302 for an identified protein may be considered as a node (e.g., row in data structure 300) which includes a field for storing a reference (i.e., pointer) value to reference a peptide in the global peptide list 310. The pointer value may serve to provide an associative linkage between the observed peptide in an indentified protein and the peptide in the global peptide list, for further processing. For example, as shown in FIGS. 3A and 3B, the reference field 304 of a node corresponding to observed peptide A, may reference node 312 in global peptide list 310 by indicating in reference field 304, a memory location value (i.e., 1947) of peptide A in global peptide list 310. It should be appreciated that other types of data structures, such as conventional arrays, may be used to associate observed peptides with matching peptides in the global peptide list, and embodiments of the invention are not limited in this respect.

We have recognized that the output of one or more searching algorithms applied to different samples of the same complex protein mixture (e.g., human plasma, mouse liver, etc.) may result in an identification of the same proteins in the different samples, albeit by using different observed peptides. Thus, in some embodiments, the sequence of a peptide in the global peptide list may be searched within the sequence of all identified proteins in the global protein list. This process may create an overall coverage map by mapping all observed peptides to potential protein targets and providing a map of all available possibilities to position a peptide within a given protein's constituent peptide list. In order to expand the list of observed peptides for a protein in a sample of a dataset, peptides not observed by the mass spectrometer analysis for the sample and protein, but occurring elsewhere in the dataset, may be added to the protein's list of observed peptides according to a process such as illustrated in FIG. 4.

In step 410, a peptide may be selected from a global peptide list comprising all observed peptides in a dataset. In some embodiments, the global peptide list may be a consolidated global peptide list having been processed according to the method illustrated in FIG. 2 and described above. In step 412, the global protein list may be searched for a protein that has a sequence containing the sequence of the peptide. In some embodiments, proteins may be identified by associative linkages created, for example, in step 250 of the process illustrated in FIG. 2. It should be appreciated however, that in other embodiments, the global protein list may be exhaustively searched to find proteins whose sequence contains the sequence of the peptide, or any other suitable search technique may be used.

In step 414, samples in which the selected protein has been identified may be determined. For a first sample and a first selected protein, it may be determined in step 416 whether the peptide is included in the protein's constituent peptide list for that sample. If the peptide is not present in the constituent peptide list, the peptide may be added to the list with a count equal to zero, in step 418. If the peptide is already present in the constituent peptide list, it may be determined whether there are any more samples which contain the selected protein in step 420. If there are more samples containing the selected protein, the process flow may return to step 414, and subsequent samples may be processed accordingly until it may be determined in step 420 that no other samples containing the selected protein are present.

After processing the first protein, it may be determined in step 422 whether additional proteins in the global protein list have a sequence containing the sequence of the peptide. If additional proteins are found, the process flow may return to step 412 where one of the additional proteins is selected, and each of the additional proteins may be processed accordingly until it may be determined in step 422 that no additional proteins containing the peptide exist in the global protein list. When no additional proteins are found, it may be determined in step 424 whether additional peptides in the global peptide list remain to be processed. If all peptides in the global peptide list have not been processed, the process flow may return to step 410 and a new peptide may be selected and processed accordingly. The above process may be repeated until all peptides in the global peptide list have been processed.

Following the process outlined in FIG. 4 and described above, any protein within any sample may have the same list of peptides, yet with different peptide counts that correspond to the actual observed number of peptide counts that were observed by the mass spectrometer. That is, a peptide having a count equal to zero represents a peptide that was not found by the mass spectrometer in a specific sample for a specific protein, but which was found somewhere else in the dataset (e.g., for the same protein in another sample).

We have further appreciated that consolidation of a dataset output from a mass spectrometer may be accomplished by dividing the dataset into subsets of nondependent groups. Each group which may be called a “similarity group,” may be a unique subset of proteins and peptides from the dataset. All of the proteins within a similarity group may have peptides in common with other proteins within that group, in any degree of relationship. By dividing the data into independent similarity groups, it may be possible to consolidate each similarity group separately rather than attempting to consolidate the dataset as a whole. Furthermore, dividing the data into similarity groups may help to reveal redundancies, as these redundancies may occur when there are proteins within the groups that can not be distinguished based on the observed constituent peptides.

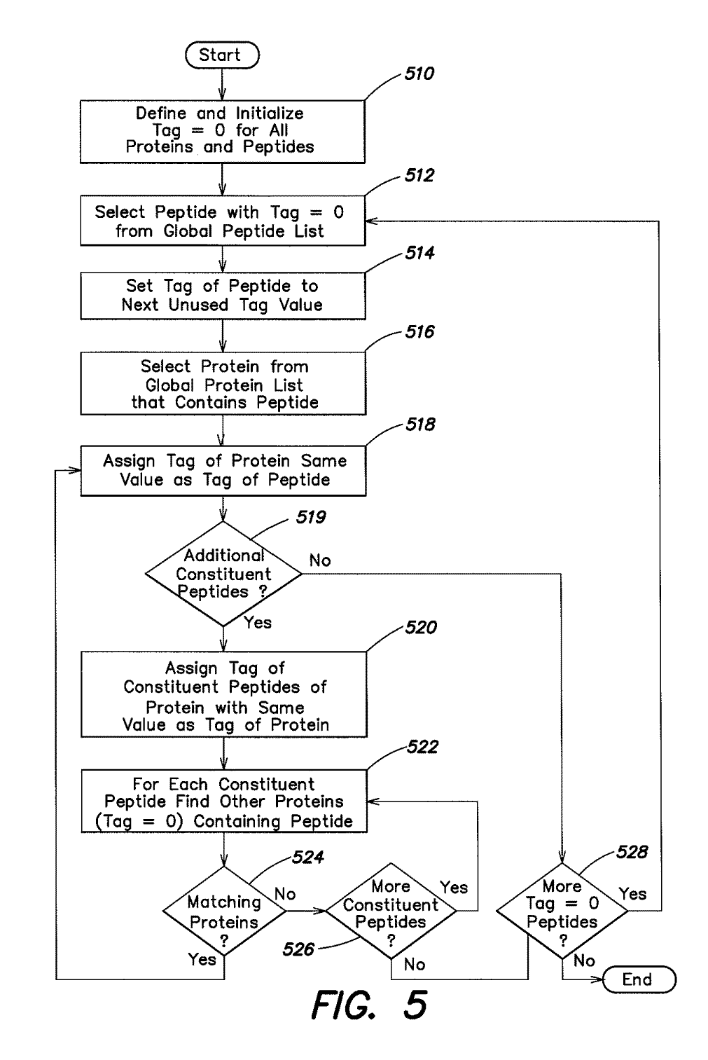

In some embodiments, a dataset may be represented as a linked list, and division of the dataset into similarity groups may be performed by labeling nodes representing proteins and constituent peptides in the dataset with “Tags,” as illustrated, for example, in FIG. 5. In step 510, each node in the linked list representing a peptide in a global peptide list or a protein in a global peptide list may define and initialize a tag by setting its value to zero. In step 512, a peptide in the global peptide list may be selected and in step 514, the tag of the peptide may be set to the next unused tag value (starting with tag=1). In step 516, a protein from the global protein list containing the peptide may be selected, and in step 518, the tag of the protein may be assigned the same value as the tag of the peptide. In step 519, it may be determined if the protein contains additional constituent peptides. If the protein does not contain additional constituent peptides, the process flow may proceed to step 528 and additional peptides may be processed. If the protein has at least one other constituent peptide, in step 520 the tag of all other constituent peptides of the protein may be assigned the same tag value as the protein. In step 522, one of the constituent peptides may be selected and a search for other proteins in the global protein list containing the peptide and having a tag value equal to zero may be performed. If any matching proteins are found, in step 524, one of the matching proteins may be selected and process flow may be returned to step 518, where the tag of the selected protein may be assigned the same value as the peptide. The process may continue in an iterative manner until no other matching proteins for a constituent peptide are found in step 524. If no matching proteins are found, it may be determined in step 526 whether there are additional constituent peptides for the currently selected protein. If there are additional constituent peptides, the process flow may return to step 522, where one of the additional constituent peptides may be selected and a search for proteins containing the additional constituent peptide may be performed. If it is determined in step 526 that all constituent peptides for a protein have been searched, the processing for the selected tag may be finished, and it may be determined in step 528 whether there are additional peptides in the global peptide list that have not yet been labeled (i.e., have tag=0). If there are such additional peptides, process flow may return to step 512, where one of the additional peptides may be selected and processed with the next unused tag value (e.g., tag=2), as described above. If it is determined in step 528 that all peptides have been labeled (i.e., no peptides remain with tag=0), the labeling process may end.

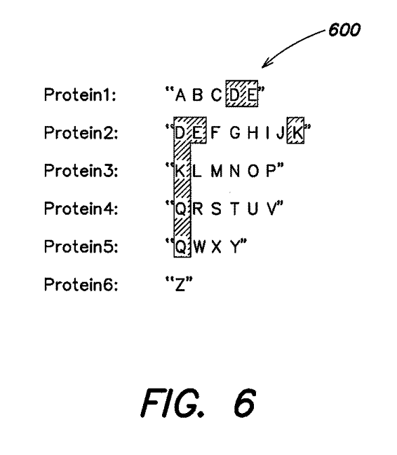

FIG. 6 depicts an illustrative example of dividing a dataset 600 into similarity groups according to the process shown in FIG. 5. In the example of FIG. 6, six proteins were identified, with each protein having at least one constituent peptide. For simplicity, each letter in FIG. 6 represents a constituent peptide. For the dataset 600, three similarity groups may be defined. Group 1 may comprise protein 1, protein 2, and protein 3, with peptides “D” and “E” being common to protein 1 and protein 2, and peptide “K” being common to protein 2 and protein 3. Group 2 may comprise protein 4 and protein 5 as peptide “Q” is common to protein 4 and 5. Group 3 may comprise protein 6 as protein 6 has no common peptides with the other proteins. It should be appreciated that the example in FIG. 6 is provided merely for illustrative purposes and is not intended to be limiting of the invention in any way.

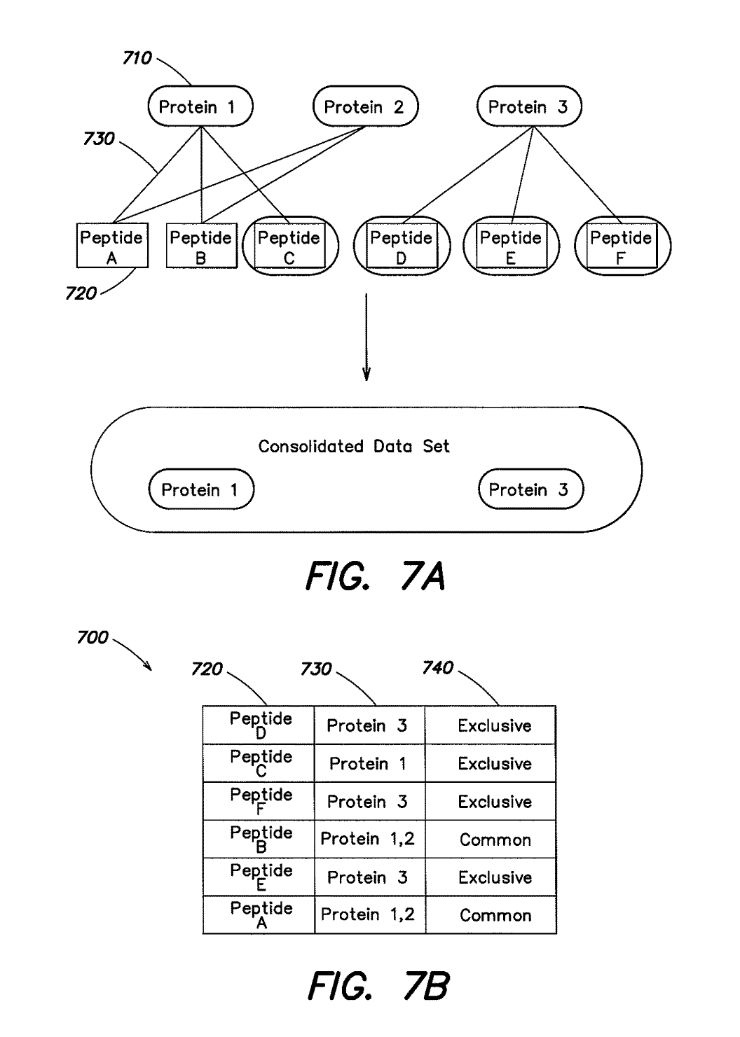

An example of representing a dataset using a linked list data structure 700 and dividing the dataset into similarity groups according to some embodiments of the invention is illustrated in FIGS. 7A and 7B. As shown in FIGS. 7A and 7B, a sample may comprise three proteins, and each of these proteins may be represented as a node 710 in a linked list 700. Each of the proteins may have constituent peptides that were observed by the mass spectrometer and were used to identify the proteins. In the linked list structure 700, the constituent peptides may also be represented as nodes 720, and referential links 730 may be formed between a protein and each of its constituent peptides, thereby defining an associative relationship between them. For example, in FIG. 7, protein 1 comprises peptide A, peptide B, and peptide C; protein 2 comprises peptide A and peptide B; and protein 3 comprises peptide D, peptide E, and peptide F. As shown in FIG. 7B, data structure 700 may comprise a plurality of fields for representing peptide nodes 720, associative links 730, and redundancy status 740. The associative links 730 in linked list structure 700 may, for example, refer to memory locations of identified proteins in the global protein list. By traversing the associative links 730 between the proteins 710 and their respective constituent peptides 720, it may be determined that protein 1 and protein 2 may belong to a first similarity group based on their common peptides A and B, and protein 3 may belong to a second similarity group due to the non-overlapping peptides observed for protein 3. Furthermore, the linked list structure 700 may be further used to consolidate the dataset as described in detail below. It should be appreciated that the example in FIG. 7 is provided merely for illustrative purposes and is not intended to be limiting of the invention in any way.

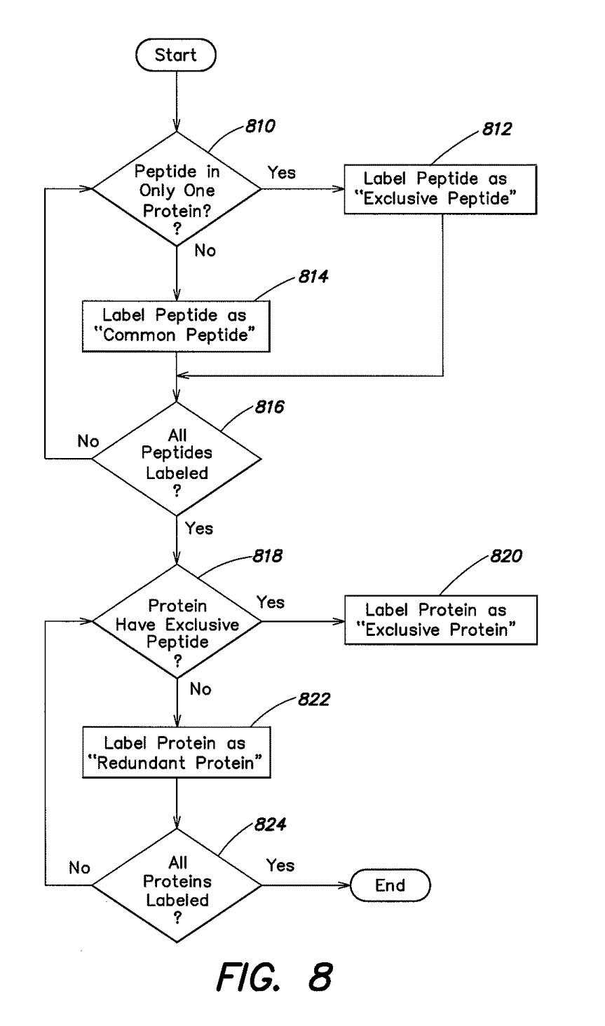

We have recognized that redundant proteins in a dataset output from a mass spectrometer may be identified and excluded based on their redundancy, resulting in a consolidated dataset. Thus, some embodiments may exclude from the dataset identical proteins (proteins with identical sequences but different names) and any other protein that is not needed to explain the observed constituent peptides. In some embodiments, peptides in a global peptide list may be labeled as exclusive or common, and proteins in a global protein list may be labeled as exclusive or redundant according to a process illustrated in FIG. 8.

In step 810, a peptide in the global peptide list may be selected and it may be determined whether the peptide is contained in one protein or more than one protein in the global protein list. In embodiments where the dataset is implemented as a linked list, this may be determined, for example, by examining the references (i.e., pointers) between the peptide and proteins in the global protein list as described in more detail below, although the associative relationship between peptides in the global peptide list and proteins in the global protein list may be determined using any suitable method. If it is determined that the peptide is only found in one protein in the global protein list, the peptide may be labeled as an “exclusive peptide” in step 812. If, on the other hand, the peptide is common to at least two proteins in the global protein list, the peptide may be labeled as a “common peptide” in step 814. The process of steps 810-814 may be repeated until it is determined in step 816 that all peptides in global peptide list have been labeled as exclusive or redundant.

Beginning in step 818, the proteins in the global protein list may be labeled. In step 818, a protein may be selected and it may be determined if any of its constituent peptides has been labeled an exclusive peptide. If at least one of the proteins constituent peptides has been labeled an exclusive peptide, the protein may be labeled as an “exclusive protein” in step 820. If however, the protein contains no constituent peptides labeled as exclusive peptides (i.e., all constituent peptides are labeled as common peptides), the protein is labeled in step 822 as a “redundant protein.” The process of steps 818-822 may be repeated until it is determined in step 824 that all proteins in the global protein list have been labeled as exclusive or redundant. In some embodiments, whether a peptide is labeled as exclusive or common, and whether a protein is labeled as exclusive or redundant may be referred to as the “redundancy status” of the peptide or protein.

Returning to the example shown in FIG. 7, traversing the associative links betweens proteins and their constituent peptides may facilitate the process of labeling peptides as exclusive or common and proteins as exclusive or redundant, and an indication of a peptide's redundancy status may be included as part of linked list data structure 700. For example, each of peptide 3, peptide 4, peptide 5, and peptide 6 may be labeled as an exclusive peptide because they are only linked to a single protein in the dataset (e.g., there is only a reference to one protein in the associative link field 730 of the linked list structure 700). In contrast peptide 1 and peptide 2 may be labeled as common peptides because they are linked to more than one protein (i.e., protein 1 and protein 2) in the dataset. Furthermore, protein 1 and protein 3 may be labeled as exclusive proteins because they contain at least one exclusive peptide, whereas protein 2 may be labeled as a redundant protein because it comprises only common peptides. After labeling each of the peptides and proteins in a dataset, the dataset (or a subset of the dataset) may be consolidated to eliminate redundant proteins (e.g., protein 2 in FIG. 7).

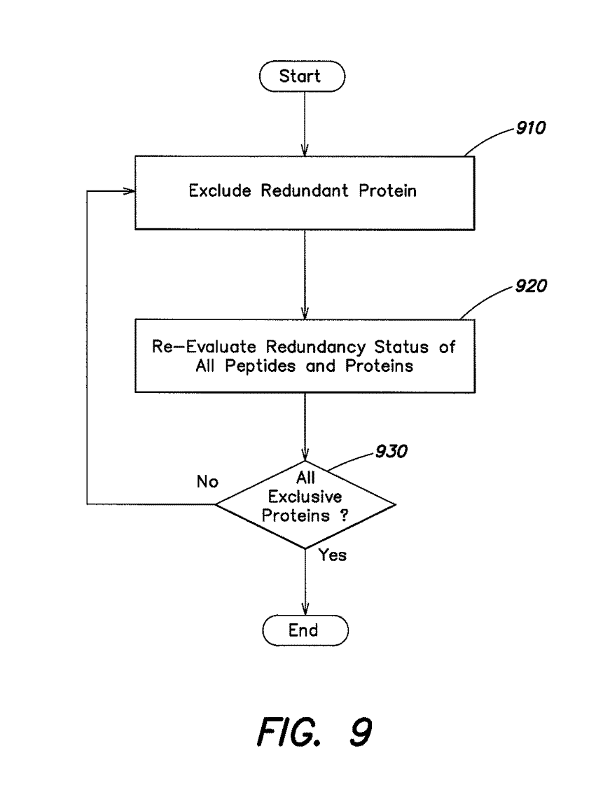

In some embodiments, an exclusion process may be used to eliminate redundant proteins in a dataset output from a mass spectrometer. The dataset may be divided into similarity groups as described above, and each similarity group may be processed separately. This may not present difficulties because exclusion of a protein that belongs to one similarity group may not affect peptides or a protein that belongs to a different similarity group. It should be appreciated however, that elimination of redundant proteins may alternatively be performed on the entire dataset rather than on similarity groups, and embodiments of the invention are not limited in this respect. In some embodiments, redundant proteins in a similarity group may be excluded according to a process as illustrated in FIG. 9, for example.

In step 910, a redundant protein may be excluded from the similarity group. In step 912, the redundancy status of all of the peptides and proteins in the similarity group may be re-evaluated. Reevaluation may be necessary because some peptides may change redundancy status from common to exclusive as a protein is excluded from the group, and some proteins may change redundancy status from redundant to exclusive as well. The process of steps 910-812 may be repeated until it is determined in step 914 that all remaining proteins in the similarity group have been labeled as exclusive proteins (i.e., all proteins contain at least one exclusive peptide).

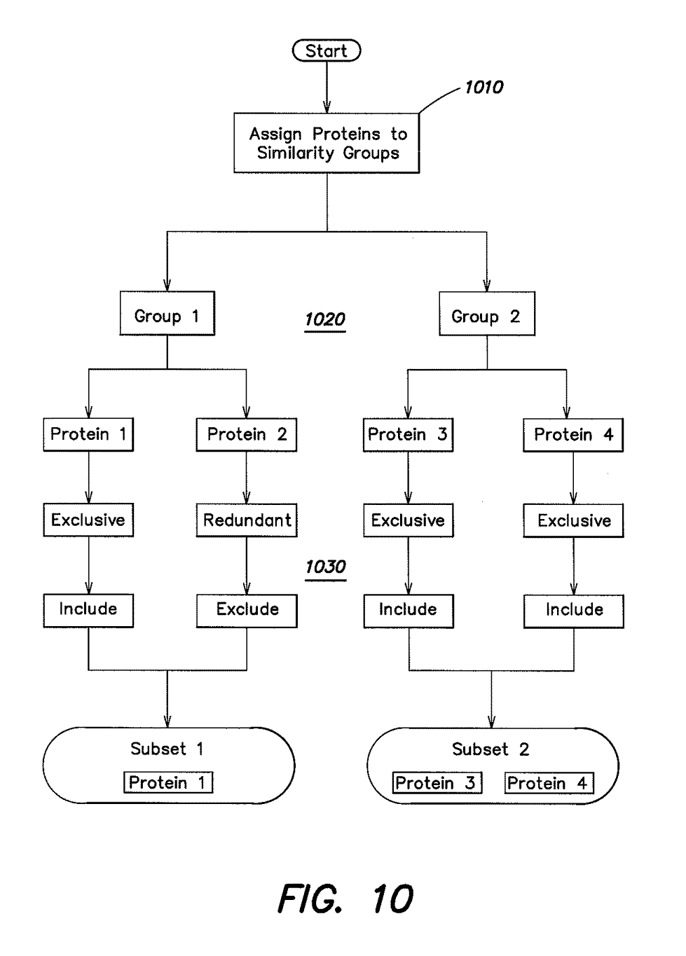

An overview of an example of a consolidation process in accordance with some embodiments is shown in FIG. 10. As described above, proteins may first be assigned to similarity groups in act 1010 based at least in part on shared common constituent peptides. In the example of FIG. 10, the four proteins 1-4 may be divided into two groups in act 1020, and then each protein may be labeled as exclusive or redundant in act 1030 according to the criteria provided above. In similarity group 1, protein 1 may be labeled as exclusive and protein 2 may be labeled as redundant. Protein 2, having been labeled as redundant, may be chosen for exclusion from the dataset resulting in subset 1 comprising only protein 1 for similarity group 1. In the example of FIG. 10, similarity group 2 may also comprise two proteins, although both proteins may be labeled as exclusive proteins, meaning that both protein 3 and protein 4 comprise at least one exclusive peptide. In this case, both proteins may be necessary for explaining all of the observed peptides. Thus, when a similarity group comprises no redundant proteins, the subset formed after the exclusion process illustrated in FIG. 8 may simply reflect the original set of proteins comprising the similarity group (e.g., subset 2 in FIG. 10).

We have appreciated that in similarity groups comprising at least two redundant proteins, switching the order in which redundant proteins are selected for elimination may produce different outcomes. Thus, in some embodiments, all possible combinations of redundant protein exclusion may be evaluated using the process illustrated in FIG. 9, and the best combination may be determined as the combination that provides a list with a minimum number of exclusive proteins. Although performing an exhaustive search of the possible combinations may in theory appear to be computationally intensive, in practice most similarity groups may contain on the order of ten proteins, so the exclusion process described herein may in most cases be processed by a standard personal computer within a few seconds. It is possible that in some circumstances, multiple combinations of redundant protein elimination in a similarity group may result in the same final minimum number of exclusive proteins.

In some embodiments, at least one heuristic decision may be used to refine the decision as to which combination is preferred when multiple combinations result in the same minimum number of exclusive proteins. For example, the heuristic decision may be based on the name of a protein, an update date for a protein in a database such as the National Center for Biotechnology Information (NCBI) database, and/or a detection quality of a peptide. In some instances, the heuristic decision may favor to exclude proteins that have names containing, “similar to,” “predicted,” “theoretical,” and/or “isoform,” as names containing these words may indicate some unreliability with the identification of the protein. It should be appreciated that other words and/or phrases may also be detected and used to exclude proteins, and embodiments of the invention are not limited in this respect.

Instead of, or in addition to, using protein names, the heuristic decision may be based in part on a protein's last modification date within the NCBI or other external proteomics database. For example, priority may be given to newer records in the database, so that proteins with older database record modification dates may be excluded first.

In yet other instances, the heuristic decision may be based in part on the detection quality of peptides in a protein. As illustrated in FIG. 1, output from a mass spectrometer typically may include an indication of the detection quality of peptides in a sample indicated as a “peptide score” value. In some instances, the heuristic decision may use the peptide score value as a basis for setting an exclusion priority for proteins, whereby proteins having peptides with lower peptide detection qualities may have a higher exclusion priority (i.e., be excluded first). Although three heuristic criteria have been set forth above, it should be appreciated that any suitable heuristics may be used to determine the order of redundant protein elimination including using multiple heuristic criteria, and embodiments of the invention are not limited in this respect.

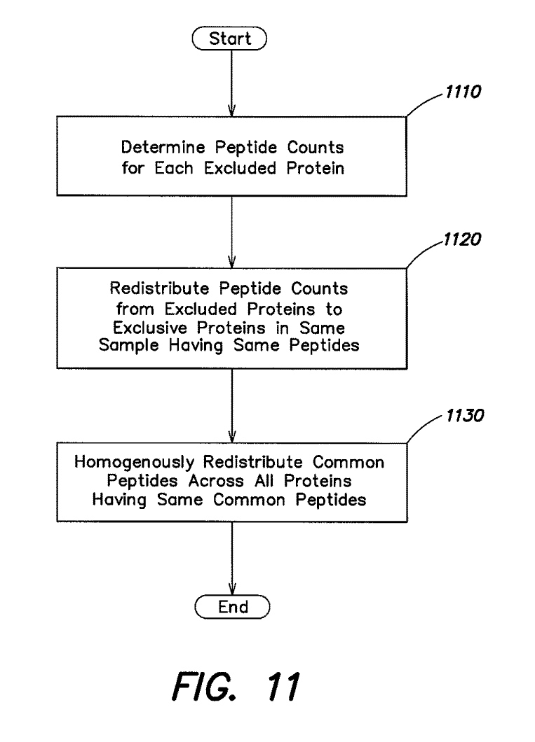

We also have recognized that by excluding redundant proteins from the global protein list in each similarity group, the random guessing process that occurs during protein identification of mass spectrometer output may be improved. By definition, each of the redundant proteins that may be excluded according to the process illustrated in FIG. 9 contain only common peptides and do not contain any exclusive peptides. Thus, the common peptides that were detected and erroneously assigned to the excluded proteins may have belonged to any of the exclusive proteins in the similarity group that also contain the common peptides. In some embodiments, a common peptide assigned to an excluded protein may be reassigned to another exclusive protein in a similarity group that also contains the common peptide. By using this approach, none of the observed peptides are lost in the exclusion process and the total peptide count may be preserved. Furthermore, we have appreciated that any common peptide (not just the common peptides of excluded proteins) may be redistributed in a homogenous manner within the group of exclusive proteins in a similarity group that contain the common peptides. In some embodiments, the redistribution of common peptides may proceed according to a process as illustrated in FIG. 11.

In step 1110, peptide counts for the common peptides in each excluded protein may be determined. In step 1120, the peptide counts for the common peptides from the excluded proteins may be redistributed to exclusive proteins having the same common peptides. Then, in step 1130, the peptide count for common peptides may be homogeneously redistributed across all proteins having the same common peptides. Peptide counts for a common peptide may be homogenously redistributed over all exclusive proteins having the common peptide because each of the exclusive proteins may have an equiprobable chance that the observed peptide corresponds to the exclusive protein. It should be appreciated that in some embodiments, steps 1110 and 1120 may be combined into a single step whereby the total peptide count for each common protein in a sample is determined (regardless of whether the common protein was identified in an exclusive or redundant peptide), and then the total peptide count may be homogenously redistributed over only exclusive proteins containing the common protein in step 1030.

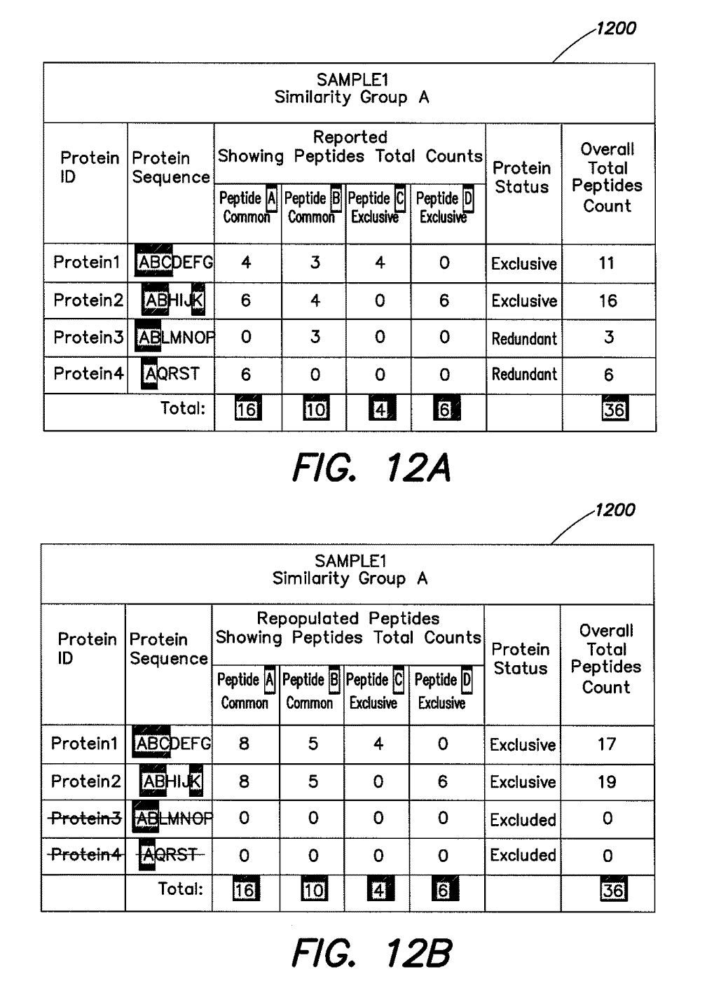

A working example of the process illustrated in FIG. 11 is shown in FIGS. 12A and 12B. In FIGS. 12A and 12B, a similarity group 1200 may comprise four proteins 1-4, with each protein having constituent peptides. In the example of FIGS. 12A and 12B, constituent peptides are represented by single letters, however, it should be appreciated that in practice, constituent peptides may be represented by a molecular sequence or by using any other suitable representation. As shown in FIG. 12A, four peptides (A, B, C, and K) were observed in a sample. Peptides C and K have been determined to be exclusive peptides, whereas peptides A and B have been determined to be common peptides. Since protein 1 contains the exclusive protein C and protein 2 contains the exclusive protein K, both protein 1 and protein 2 may be required to explain the observed peptides. However, protein 1 and protein 2 also contain the observed common peptides A and B, thereby allowing protein 3 and protein 4 to be classified as redundant (i.e., they contain no exclusive peptides). After exclusion of proteins 3 and 4 from the similarity group, the peptide count of 6 observed for peptide A and originally assigned to protein 4, and the peptide count of 3 observed for peptide B and originally assigned to protein 3 may be redistributed to exclusive proteins 1 and 2 which also contain peptides A and B. The redistribution (i.e., repopulation) of peptides A and B may result in the peptide counts shown in FIG. 12B. As shown in FIG. 12B, the total peptide count for each of common peptide A and common peptide B may remain the same after redistribution, and the peptide counts may homogeneously distributed over the non-excluded exclusive proteins 1 and 2.

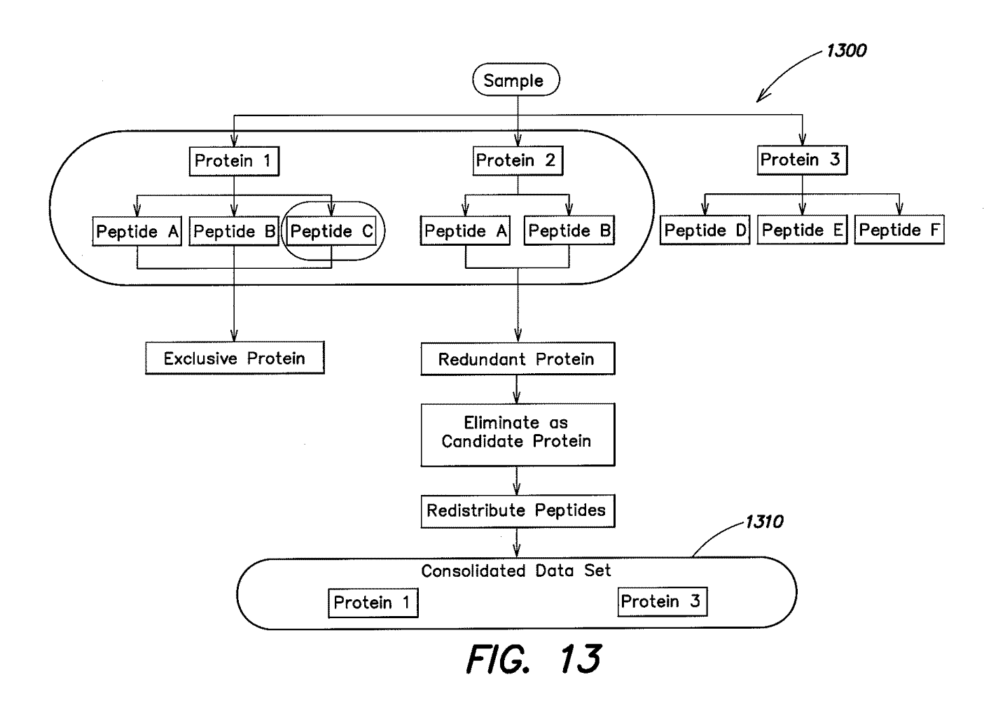

A schematic overview of a consolidation process according to some embodiments is illustrated in FIG. 13. Output from a mass spectrometer may be cross-referenced with a database using a searching algorithm to construct a list of proteins and constituent peptides. In the example of FIG. 13, a dataset 1300 comprises three proteins which may be divided into two similarity groups based on the proteins' shared common peptides. Within each similarity group, each protein may be labeled as exclusive or redundant based at least in part on whether or not the protein comprises any exclusive peptides. Redundant proteins may then be systematically eliminated from the similarity group until all remaining proteins in the group are classified as exclusive proteins. Following exclusion, common peptides in the similarity group may be redistributed to preserve the total peptide count and to homogenously distribute the common peptides among their parent proteins, resulting in a consolidated data set 1310. The consolidation of mass spectrometer output as disclosed herein may filter out proteins that are not necessary to explain the peptides observed across multiple samples, resulting in a dataset that effectively may have a higher signal-to-noise ratio compared to the original dataset 1300.

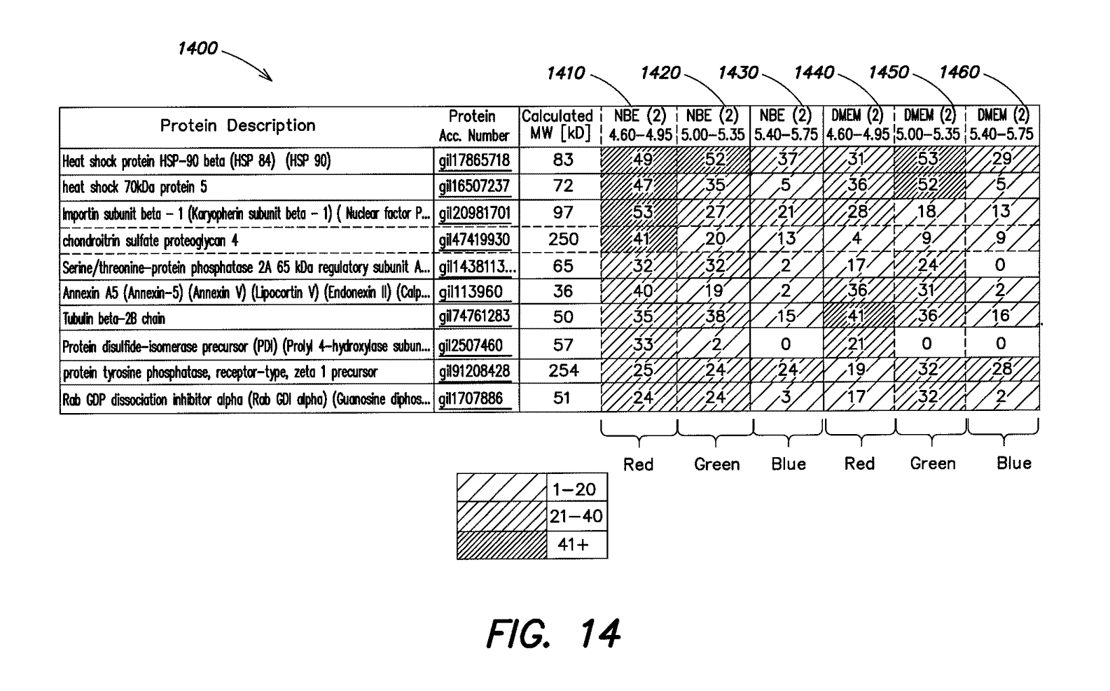

Some embodiments are directed to methods and apparatus for formatting and displaying mass spectrometer data. In some embodiments, observed constituent peptides from a mass spectrometry experiment may be displayed in a data structure similar to that shown in FIG. 14 at 1400, which may correspond to a data structure in an underlying computer-readable medium not shown. In general, it should be understood that for such data displays discussed herein, there is an underlying computer readable data structure populated with the information from which the display is generated. Such computer implemented data structures may be implemented in any suitable form, such as, but not limited to, a relational database, linked lists, arrays, matrices, a flat file database, and so forth.

In some embodiments, observed peptides may be separated into bins based at least in part on at least one molecular property of each peptide. Non-limiting examples of molecular properties may include any molecular property output from a mass spectrometer such as an isoelectric point, a molecular weight, or a measure of hydrophobicity. In the example of FIG. 14, the observed peptides have been categorized according to their characteristic isoelectric point, although it should be appreciated that any other suitable measure, including those mentioned above and others, may be used. According to the example illustrated in FIG. 14, a data structure display 1400 may be a table having multiple columns, with each column representing a different pH range. For example, column 1410 may represent a pH range from 4.60-4.95, column 1420 may represent a pH range from 5.00-5.35, and column 1430 may represent a pH range from 5.40-5.75. The data structure may also have rows representing individual proteins identified in part based on the observed peptides. The intersection of the rows and columns in data structure display 1400 may define a plurality of cells in which peptide counts may be represented. According to the example of FIG. 14, each cell may contain the total number of peptides observed within a predefined pH range (e.g., as defined by the columns) and assigned to a particular protein (e.g., as defined by the rows). Although the total number of peptides is shown in each cell of FIG. 14, alternate measured quantities may also be used, including, but not limited to, the number of unique peptides observed for a protein or the percent coverage of peptides for a protein.

In some embodiments, for each cell, the peptide count may represent the number of peptides observed in one sample of a mass spectrometer experiment, the total number of peptides observed across multiple repeated samples, and/or an average number of peptides observed across multiple repeated samples. Additionally, a first set of columns may represent a first group of samples and a second set of columns may represent a second group of samples. For example, columns 1410, 1420, and 1430 may represent the mass spectrometer output for a liver sample, and columns 1440, 1450, and 1460 may represent the mass spectrometer output for a liver sample treated with a pharmaceutical compound. Having different columns represent different experimental conditions may facilitate, for example, a visual assessment of pre- and post-treatment effects of a pharmaceutical compound on a complex protein sample such as a blood or tissue sample, or a visual assessment of two (or more) different treatments on the sample. Although the data structure display 1400 has been described as having columns which represent ranges of pH and rows which represent different proteins, it should be appreciated that some embodiments of the invention may alternatively interchange the functionality of rows and columns such that columns may represent different proteins and rows may represent ranges of pH.

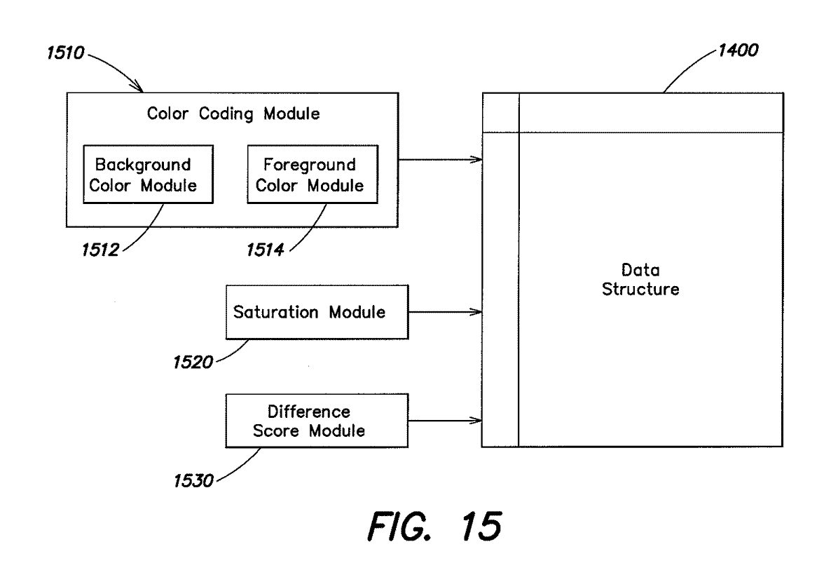

Additionally, embodiments may comprise at least one module to assign one or more visual parameters to the columns or rows of data structure display 1400 to emphasize certain features of the data. Accordingly, some embodiments may comprise a color coding module 1510, and/or a saturation module 1520 as shown in FIG. 15. In some embodiments, the color coding module may further comprise a background color module 1512 and a foreground color module 1514 for coding cells using background colors and foreground colors, respectively. Foreground colors may be used, for example, to represent cells having a value greater than zero, and background colors may be used to represent cells having a value equal to zero. In some embodiments, background colors may be used to visually differentiate different experimental conditions or “cases.” Using the illustrative example of FIG. 14, columns 1410, 1420, and 1430 may represent mass spectrometer output from one case, and columns 1440, 1450, and 1460 may represent mass spectrometer output from another case. Each cell having a value equal to zero in columns 1410, 1420, and 1430 may be coded with a first background color to indicate that the cell belongs to the first case, and each cell having a value equal to zero in columns 1440, 1450, and 1460 may be coded with a second background color to indicate that the cell belongs to the second case. In some embodiments, the background colors may be faint (nearly-white) colors so as not to distract from the cells having non-zero values, and the background colors may be selected based on a first spectrum or color model.

In some embodiments, at least one foreground color may be used to color code cells in the axis representing ranges of pH. For example, if ranges of pH are represented as columns in data structure display 1400 as shown in FIG. 14, the cells in a column may be color coded from lowest pH to highest pH or vice versa using a second spectrum or color model. The second spectrum or color model selected for coding foreground colors may be different than the first spectrum or color model selected for coding background colors so as to allow for differentiation of different cases (represented by zero-value cells and background colors) and different ranges of pH (represented by foreground colors). Non-limiting examples of spectra/color models include a conventional pH spectrum (e.g., red (acidic)→green (neutral)→blue (basic)) an RGB color model, a CMYK color model, or any other spectrum/color model. For example, cells in columns 1410 and 1440 may be color coded red, cells in columns 1420 and 1450 may be color coded green, and cells in columns 1430 and 1460 may be color coded blue, as indicated in FIG. 14.

In some embodiments, saturation module 1520 may code the magnitude of the value in each cell of data structure display 1400 by modifying the saturation value of the foreground color used for each cell. For example, cells with a large number of observed peptides may have a foreground color with greater saturation value than cells with a small number of observed peptides or vice versa. Distinguishing the cells of data structure display 1400 based on saturation value, may facilitate the rapid identification of regions of data structure display 1400 which show the largest number of observed peptides (i.e., cells that have the highest expression levels). In the example of FIG. 14, saturation levels are indicated as cross-hatching in each of the cells of data structure display 1400. For simplicity, the saturation levels are illustrated in three ranges with different densities of cross hatching. Cells with larger values may have dense cross-hatching which may represent darker colors, whereas cells with smaller values may have sparse cross-hatching which may represent lighter colors. While three ranges of saturation values are illustrated in FIG. 14, it should be appreciated that any level of saturation granularity, including a granularity of one, may be represented in data structure display 1400 by saturation module 1520, and embodiments of the invention are not limited in this respect.

Instead of, or in addition to, using color to visually highlight various cells of data structure display 1400, visual properties of the text in each cell may be modified. In some embodiments, the text color, font, and/or style (e.g., bold, underline, italics, etc.) may be altered to provide additional visual information. For example, the cell having the highest expression level (largest magnitude value) in each row of data structure display 1400 may be shown in bold to emphasize this feature of the data. It should be appreciated that any combination of color and textual appearance coding may be used, and the examples provided above are not meant to limit embodiments of the invention in any way.

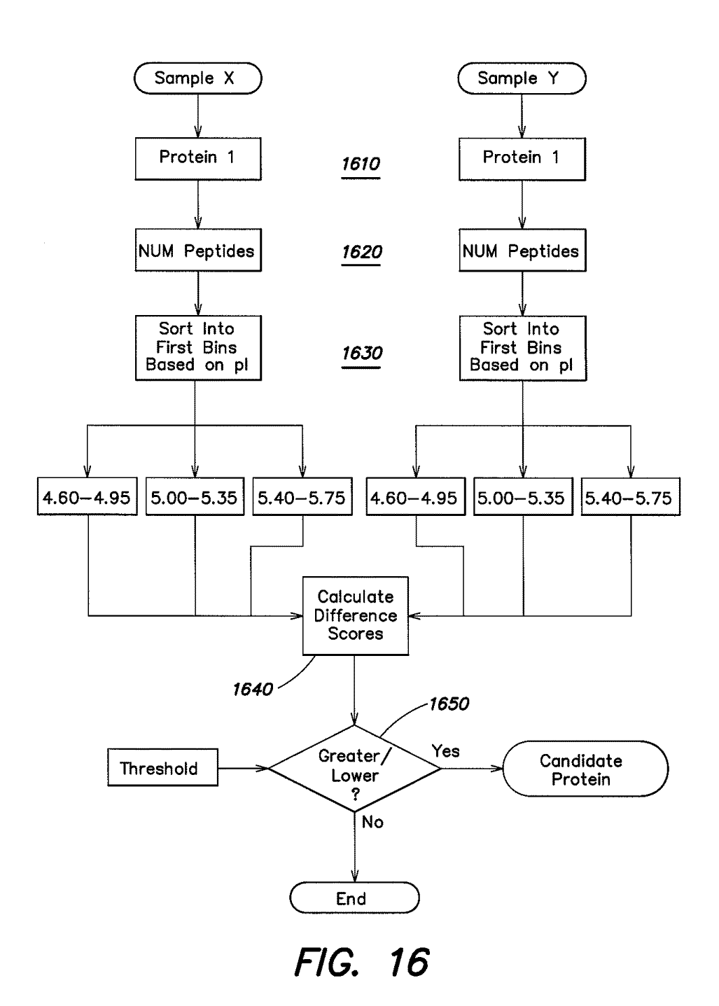

We have appreciated that some mass spectrometry experiments may be designed to determine the effect of a pharmaceutical compound on a complex protein mixture such as blood or tissue. Thus, some embodiments may additionally comprise a difference score module 1530 for calculating differences in the number of observed peptides for different experimental conditions. For example, as shown in FIG. 14, columns 1410, 1420, and 1430 may represent mass spectrometer output for a liver sample, and columns 1440, 1450, and 1460 may represent mass spectrometer output for a liver sample treated with a pharmaceutical compound. Difference score module 1530 may calculate differences between columns for two or more experimental conditions for the same range of pH. In some embodiments, if the difference score for a protein in a pH range is greater than a threshold value, then the protein may be labeled as a candidate protein for further investigation. According to some embodiments, difference score module 1430 may operate according to a process as shown in FIG. 16, for example.

According to the example of FIG. 16, a mass spectrometry experiment may comprise two samples. For example, sample X may be an untreated liver sample from a rat, and sample Y may be a sample from the same liver, but treated with a pharmaceutical compound. Both samples may be subjected to mass spectrometry and the output may reveal in step 1610 that both samples contain a protein 1. In step the number of observed peptides corresponding to protein 1 for each sample may be determined. In step 1630, the observed peptides for the first sample may be sorted into a first set of bins based on each peptide's isoelectric point (pI), and the observed peptides for the second sample may similarly be sorted into a second set of bins based on pI. Although pI has been chosen as an exemplary molecular property for sorting the observed peptides, it should be appreciated that any other suitable molecular property including, but not limited to, molecular weight and/or a measure of hydrophobicity, may also be used. The peptide counts for the first sample and the second sample for protein 1 may be organized into columns (or rows) in data structure display 1400 as illustrated in FIG. 14, lined for color saturation. Difference score module 1530 may calculate difference scores between values in the first bins and the second bins in step 1640, and the differences scores may be compared to a predetermined threshold in step 1650. Differences relative to the threshold may be labeled or otherwise highlighted in some manner to indicate that further investigation of the protein may be warranted (i.e., because the pharmaceutical compound appears to have an effect on the sample). In some embodiments, some or all of the calculated difference scores may be presented as text on a display. It should be appreciated that any difference score formula may be used to calculate difference scores, including, but not limited to, weighted or unweighted subtraction of values in the first bins and second bins, and embodiments of the invention are not limited in this respect.

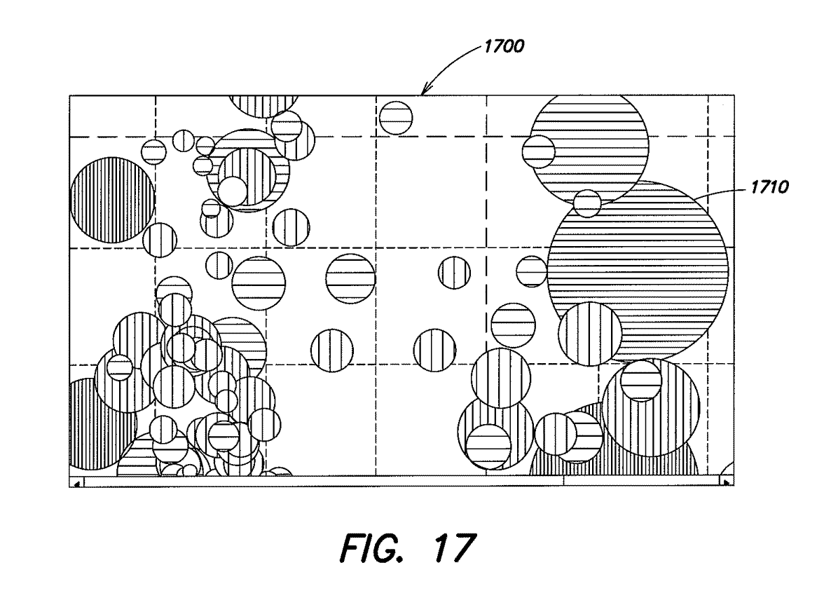

In addition to a textual representation of difference scores, difference scores may be represented graphically in a variety of ways according to some embodiments of the invention. For example, difference scores may be represented as a bubble chart 1700 shown in FIG. 17. In the example of FIG. 17, each circle 1710 may represent a protein, and one axis of the bubble chart 1700 may represent pI, whereas the other axis may represent molecular weight. In some embodiments, the size of each of the circles 1710 may represent a difference score for a pair of cells in data structure display 1400. Additionally, each of the circles 1710 may be color coded by color coding module 1510 so that one color may represent a positive difference score and another color may represent a negative difference score. In some embodiments, the colors used to code the circles 1710 in the bubble chart 1700 may be derived from the background colors used to code different cases in data structure display 1400. For example, the background color used in data structure display 1400 to represent case 1 (e.g., untreated liver sample) may be blue and the background color used to represent case 2 (e.g., treated liver sample may be red. If positive difference scores are defined as observing more peptides in case 2 versus case 1 for a particular protein, positive difference scores may be coded as red, whereas negative difference scores may be coded as blue. It should be appreciated that any colors may be used to code positive and/or negative difference scores and embodiments of the invention are not limited in this respect.

In some embodiments, saturation module 1520 may be used to assign different saturation values to one or more circles 1710 in the bubble chart 1700, so that larger difference scores may be represented by more saturated (i.e., darker) circles whereas smaller difference scores may be represented by less saturated (i.e., lighter) circles or vice versa. In some embodiments, pI may be represented on the horizontal axis of the bubble chart with molecular weight represented on the vertical axis, and in at least one other embodiment, pI may be represented on the vertical axis of the bubble chart 1700 with molecular weight represented on the horizontal axis.

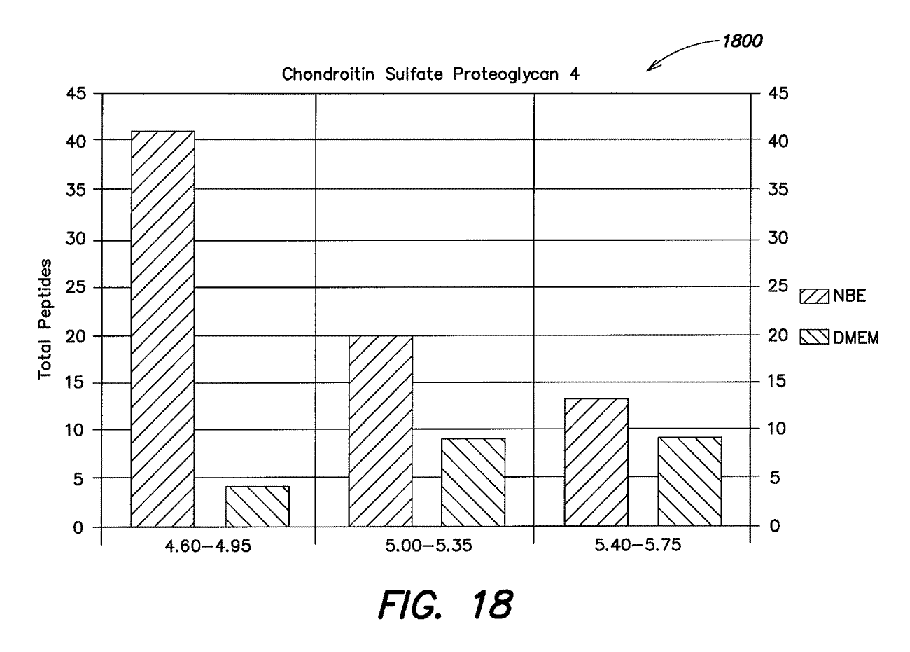

In some embodiments, difference scores for at least one protein may be represented as a bar graph 1800 shown in FIG. 18. Bar graph 1800 may be divided into sections based on different ranges of pH or different cases (i.e., different experimental conditions) as defined in data structure display 1400. Bar graph 1800 is divided into three pH ranges corresponding to the ranges illustrated in FIG. 16. In some embodiments, the vertical axis of the bar graph 1800 may be the total number of peptides observed within a pH range. Within each pH range, the total number of observed peptides may be plotted for samples having different experimental conditions as shown in FIG. 18, in which the difference score may be interpreted as the difference in the height of the bars in each pH region. Alternatively, the difference score itself may be plotted as a single bar in each pH region.

The bars in each region may be color coded or otherwise distinguished from each other, thereby identifying to which sample (i.e., case) or pH range each bar corresponds. In some embodiments, the colors used to code the bars in the bar graph 1800 may correspond to foreground colors or background colors used in data structure display 1400. For example, in embodiments in which the bar graph represents the total number of peptides in each range of pH, as illustrated in FIG. 18, the color of the bars may correspond to the background colors used in data structure display 1400 to represent the different cases. In other embodiments in which the bar graph 1800 is divided into sections based on case, the color of the bars in the bar graph may correspond to the foreground colors used in data structure display 1400 to represent the different pH ranges. In yet other embodiments, both foreground colors and background colors used in data structure display 1400 may be represented in the bar graph 1800, so that the color of bars may be coded as described above and the background of each of the sections may be coded with the color scale not used to code the bars. For example, if the background colors (representing case) are used to code the bars as illustrated in FIG. 18, the foreground colors (representing pH range) may be used to color code the backgrounds of each of the sections in the bar graph 1800.

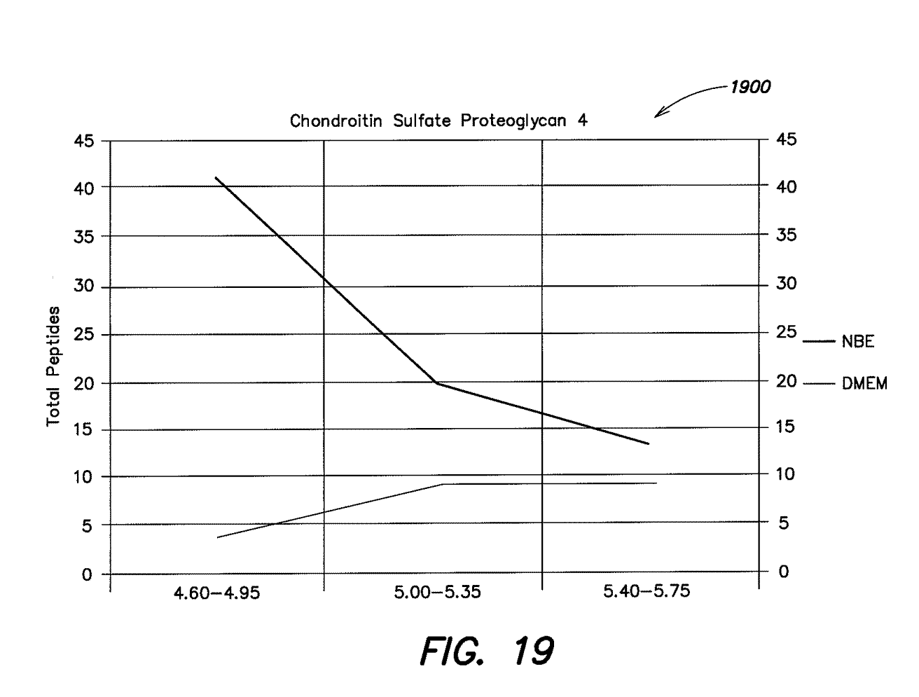

In some embodiments, difference scores for at least one protein may be represented as a line graph 1900 shown in FIG. 19. As with the bar graph 1800, the line graph 1900 may represent the total number of peptides observed in at least one of the pH ranges defined in data structure display 1400. Difference scores may be interpreted as the difference in the height of the lines plotted in the middle of each pH region. In some embodiments, color coding may be performed by color coding module 1510 in a similar manner as described for the bar graph 1800.

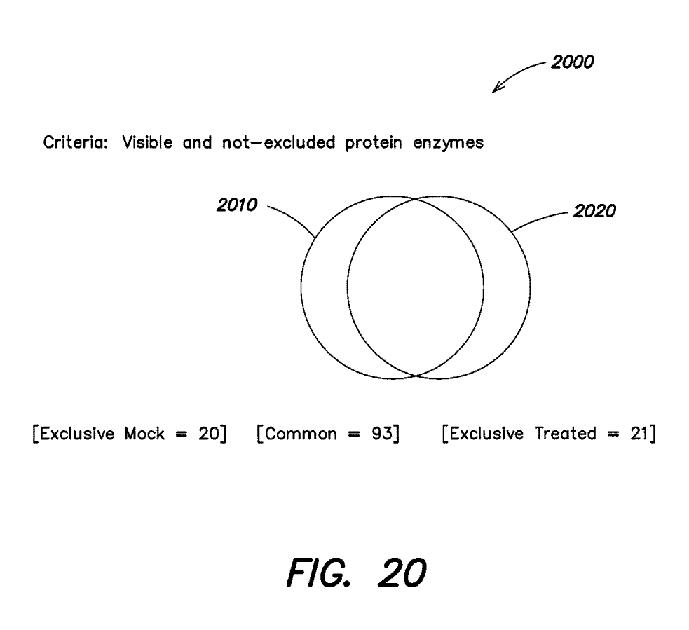

In some embodiments, a difference in the number of exclusive proteins in samples under different experimental conditions may be displayed using a Venn diagram 2000 illustrated in FIG. 20. For example, the circle 2010 may represent the number of exclusive proteins in one sample (e.g., control sample of liver), whereas the circle 2020 may represent the number of exclusive proteins in another sample (e.g., treated sample of liver), and the degree of overlap may represent the number of exclusive proteins common to both samples. In some embodiments, the color of the circles in the Venn diagram 2000 may be color coded using background colors or foreground colors used in data structure 1400 to indicate the different samples (i.e., cases) and/or ranges of pH).

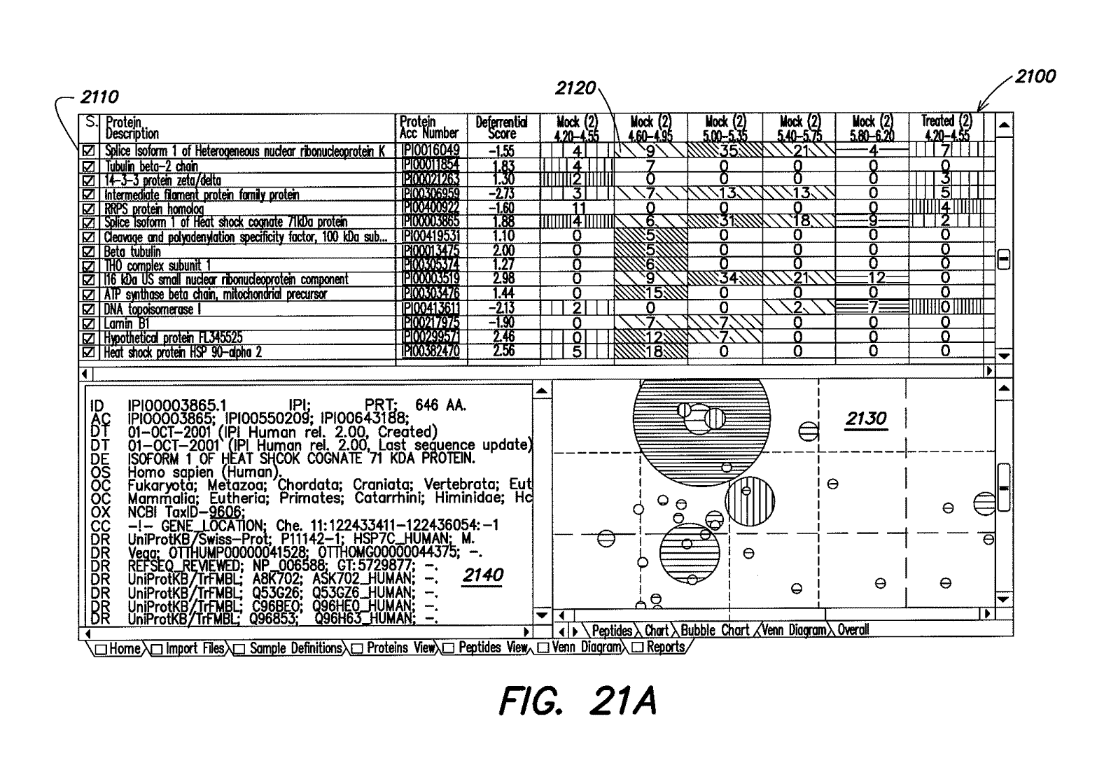

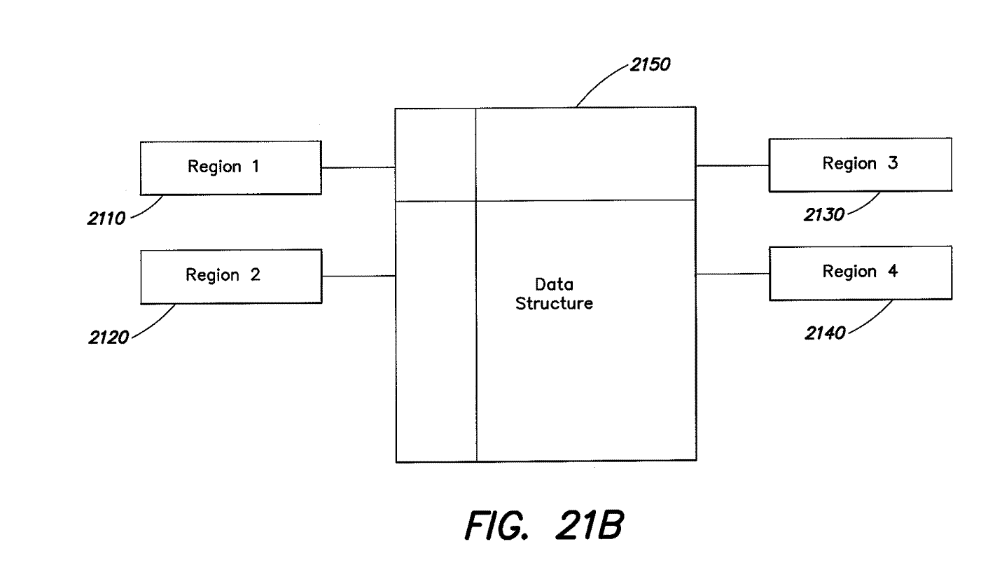

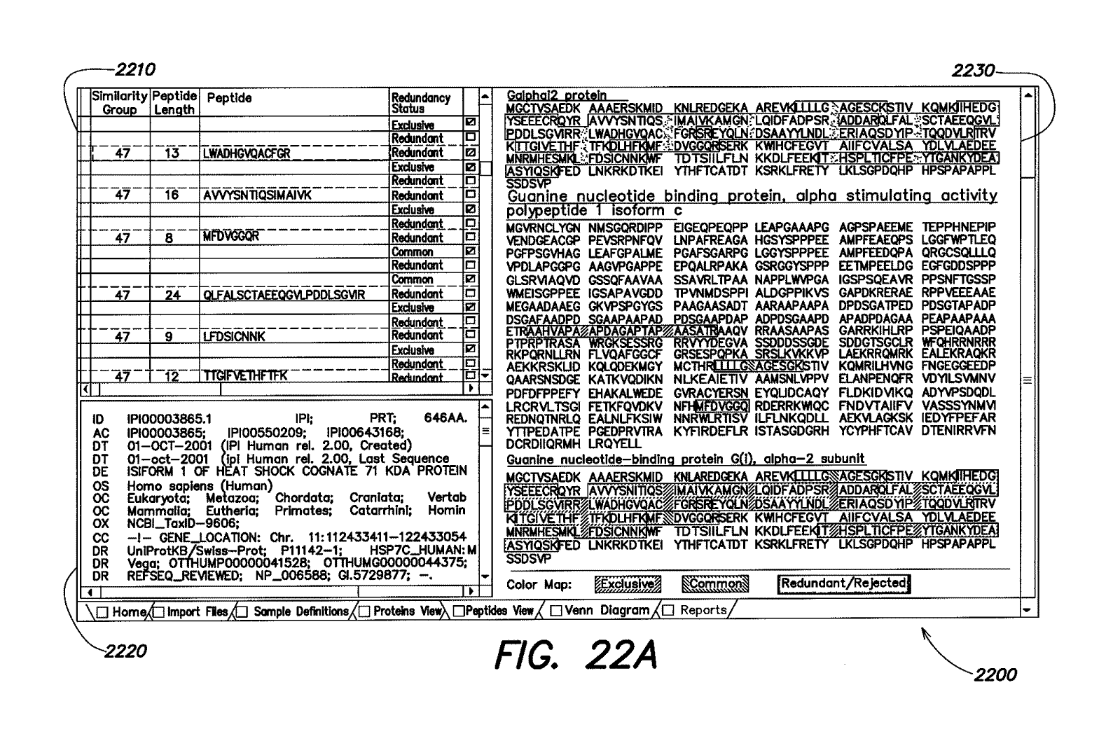

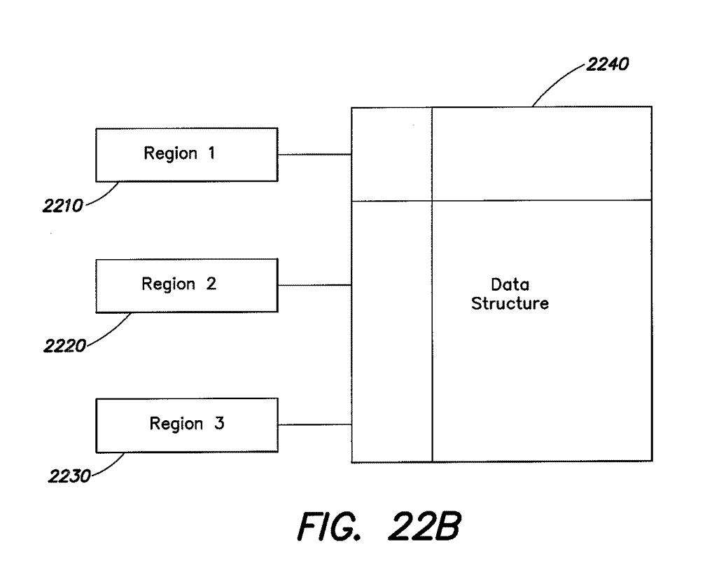

One or more embodiments described above, or portions of one or more of them, may be combined and implemented as a display, produced by a software program, or portion thereof, as illustrated in FIGS. 21 and 22. For example, such a display may comprise multiple regions, and some or all of the regions may be configured to interact. We have recognized and appreciated that interactivity between regions of a display produced by a software program(s) as disclosed herein, may provide a rapid identification and verification method for analysis of mass spectrometer data previously not available with existing mass spectrometer analysis techniques.

In the example of FIG. 21A, a display 2100 may have four regions 2110-2140, and each of the regions may provide different but complementary information about the results of a mass spectrometry experiment. A first region 2110, located in the upper left of display 2100, may provide technical and/or general information about proteins identified by the mass spectrometer. For example, the first region 2110 may include information about a protein's name, description, accession number, molecular weight, pI, etc. A second region 2120, located in the upper right of display 2100 may comprise a data structure display similar to data structure display 1400 shown in FIG. 14. As described above, data structure display 1400 may comprise data corresponding to observed peptide counts in one or more samples of a mass spectrometry experiment, and various visual parameter values may be assigned to cells within data structure display 1400 to highlight particular features of the data structure. A third region 2130, located in the lower right of display 2100 may comprise at least one visual representation of difference scores as defined above. In the example of FIG. 21A, a bubble chart is shown in region 2130, however, it should be appreciated that any visual representation that provides information related to protein validation may be used. A fourth region 2140, located in the lower left of display 2100 may comprise detailed information about a selected protein. The information displayed in the fourth region 2140 may, for example, be gathered from a publically available database such as the NCBI database or the Swiss-Prot database.

In some embodiments, each of the regions of display 2100 may be associated with a user-configurable window, the size which may be changed. In one embodiment, if the size of a window is reduced below a predefined minimal size, the window (and all information contained therein) may be hidden from view. Windows corresponding to regions of display 2100 may also, in some embodiments, be minimized, maximized or removed. Additionally user-configurable properties defined for one region of display 2100 may be transferred to other regions as well. For example, a user may select one or more proteins to be hidden from the list of proteins shown in the first region 2110. Accordingly, the circles in the bubble chart shown in the third region 2130 which correspond to the “hidden” proteins may also not be displayed.

In some embodiments, the combination of multiple regions, as illustrated in FIGS. 21A and 21B, may impart additional functionality beyond the functionality of each area when considered individually. For instance, different regions may be configured to interact so that a selection of an object in one region may affect the display of information in some or all of the other regions. In some embodiments, this may be accomplished by linking some or all of the regions to a common data structure 2150, as shown in FIG. 21B, thereby allowing synchronous updates of each of the linked regions. For example, output from a mass spectrometry experiment may be represented as shown in display 2100, and a user may want to display more information about a protein corresponding to a circle in the bubble chart having the largest diameter (e.g., showing the largest difference score). In some embodiments, the user may select the largest circle in region 2130 using a mouse or any other suitable computer input device, and in response to the selection, the corresponding protein in the list of proteins displayed in the first region 2110 may be highlighted, the corresponding cells and/or row in the data structure displayed in the second region 2120 may be highlighted, and/or the detailed protein information in the fourth region 2140 may be updated. Information in the fourth region 2140 may be updated, for example, by querying a public database for information about a protein corresponding to the accession number of the highlighted protein in the first region 2110. Similarly, selecting a protein from the list of proteins in the first region 2110 may affect the information displayed in one or more of the other regions.

Peptide information calculated in accordance with some embodiments may also be integrated as a display 2200 produced by a portion of a software program as illustrated in FIG. 22A. In a first region 2210 of display 2200, peptide information may be displayed. The peptide information may include characteristic peptide information such as the peptide's molecular sequence or peptide length (i.e., number of amino acids), or derived peptide information such as the peptide's redundancy status or a similarity group to which the peptide belongs. A second region 2220 may display protein information gathered from a publicly available database, and this information may be updated, for example, upon selection of a different peptide in the peptide list shown in the first region 2210. A third region 2230 may display a molecular sequence of at least one identified protein, and molecular sequence of observed peptides may be highlighted in the molecular sequence of the displayed proteins. In some embodiments, the redundancy status of the highlighted observed peptides may be color coded to facilitate their identification in the molecular sequence of the proteins. In some embodiments, the first region 2210, the second region 2220, and the third region 2230 may be linked to a common data structure 2240 to facilitate updates to one or more of the regions of display 2200 in a similar manner as described above for display 2100.

In some embodiments, displays produced by portions of a software program, such as the displays illustrated in FIGS. 21A and 22A, may be produced by the same software program and they may be linked to a common underlying data structure comprising data derived according to various embodiments disclosed herein. While display 2100 comprises four regions 2110-2140, and display 2200 comprises three regions 2210-2230, it should be appreciated that any number of regions may be used in any display produced by a portion of a software program, and the provided examples do not limit the invention in any way.

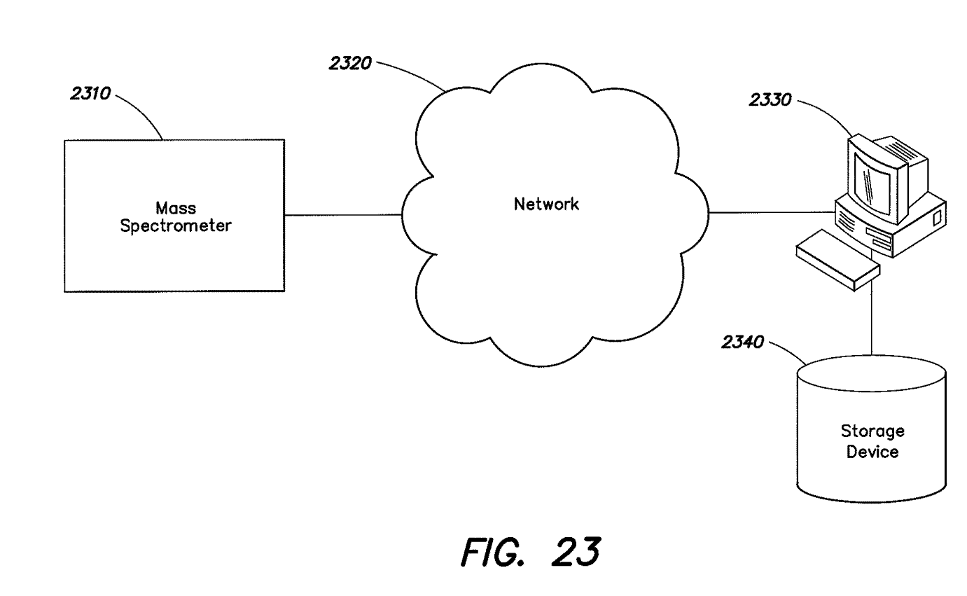

FIG. 23 illustrates an exemplary system on which some embodiments may be employed. The system comprises a mass spectrometer 2310 which analyzes one or more samples to determine the content of the samples as described above. In some embodiments, the mass spectrometer 2310 is connected to a computer 2330 via a network 2320 such as a wired or wireless local area network (LAN) or a wide area network (WAN) such as the Internet. Output from the mass spectrometer 2310 may be transmitted to the computer 2330 via the network 2320 for further analysis. In other embodiments, the computer 2330 and the mass spectrometer 2310 are not connected via a network, and the output of the mass spectrometer may be transferred to the computer 2330 via a portable storage device, such as a flash drive or a compact disc. In addition to any internal storage devices that computer 2330 may have, the exemplary system shown in FIG. 23 may also comprise a storage device 2340 connected to computer 2330, which stores data and/or various programs to be executed on computer 2330.

It should be appreciated that the computer system illustrated in FIG. 23 is merely one example of a computer system on which some embodiments may be employed, and embodiments may also be used with other types of computer systems employing any number of computers, networks, and storage devices connected in any suitable configuration.

Having thus described several aspects of some embodiments of this invention, it is to be appreciated that various alterations, modifications, and improvements will readily occur to those skilled in the art. Such alterations, modifications, and improvements are intended to be part of this disclosure, and are intended to be within the spirit and scope of the invention. Accordingly, the foregoing description and drawings are by way of example only.

The above-described embodiments of the present invention can be implemented in any of numerous ways. For example, the embodiments may be implemented using hardware, software or a combination thereof. When implemented in software, the software code can be executed on any suitable processor or collection of processors, whether provided in a single computer or distributed among multiple computers.

Further, it should be appreciated that a computer may be embodied in any of a number of forms, such as a rack-mounted computer, a desktop computer, a laptop computer, or a tablet computer. Additionally, a computer may be embedded in a device not generally regarded as a computer but with suitable processing capabilities, including a Personal Digital Assistant (PDA), a smart phone or any other suitable portable or fixed electronic device.

Also, a computer may have one or more input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets. As another example, a computer may receive input information through speech recognition or in other audible format.

Such computers may be interconnected by one or more networks in any suitable form, including as a local area network or a wide area network, such as an enterprise network or the Internet. Such networks may be based on any suitable technology and may operate according to any suitable protocol and may include wireless networks, wired networks or fiber optic networks.

Also, the various methods or processes outlined herein may be coded as software that is executable on one or more processors that employ any one of a variety of operating systems or platforms. Additionally, such software may be written using any of a number of suitable programming languages and/or programming or scripting tools, and also may be compiled as executable machine language code or intermediate code that is executed on a framework or virtual machine.

In this respect, the invention may be embodied as a computer readable medium (or multiple computer readable media) (e.g., a computer memory, one or more floppy discs, compact discs, optical discs, magnetic tapes, flash memories, circuit configurations in Field Programmable Gate Arrays or other semiconductor devices, or other tangible computer storage medium) encoded with one or more programs that, when executed on one or more computers or other processors, perform methods that implement the various embodiments of the invention discussed above. The computer readable medium or media can be transportable, such that the program or programs stored thereon can be loaded onto one or more different computers or other processors to implement various aspects of the present invention as discussed above.

The terms “program” or “software” are used herein in a generic sense to refer to any type of computer code or set of computer-executable instructions that can be employed to program a computer or other processor to implement various aspects of the present invention as discussed above. Additionally, it should be appreciated that according to one aspect of this embodiment, one or more computer programs that when executed perform methods of the present invention need not reside on a single computer or processor, but may be distributed in a modular fashion amongst a number of different computers or processors to implement various aspects of the present invention.

Computer-executable instructions may be in many forms, such as program modules, executed by one or more computers or other devices. Generally, program modules include routines, programs, objects, components, data structures, etc. that perform particular tasks or implement particular abstract data types. Typically the functionality of the program modules may be combined or distributed as desired in various embodiments.

Also, data structures may be stored in computer-readable media in any suitable form. For simplicity of illustration, data structures may be shown to have fields that are related through location in the data structure. Such relationships may likewise be achieved by assigning storage for the fields with locations in a computer-readable medium that conveys relationship between the fields. However, any suitable mechanism may be used to establish a relationship between information in fields of a data structure, including through the use of pointers, tags or other mechanisms that establish relationship between data elements.

Various aspects of the present invention may be used alone, in combination, or in a variety of arrangements not specifically discussed in the embodiments described in the foregoing and is therefore not limited in its application to the details and arrangement of components set forth in the foregoing description or illustrated in the drawings. For example, aspects described in one embodiment may be combined in any manner with aspects described in other embodiments.

Also, the invention may be embodied as a method, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

Further, it should be appreciated that some embodiments may be integrated with a mass spectrometer, whereas other embodiments may receive data output from a mass spectrometer and may process the data separately from the mass spectrometer. That is, output from a mass spectrometer may be received either directly or indirectly from the mass spectrometer. For example, in some embodiments, data from a mass spectrometer may be transmitted via a network to a computer on which one or more of the embodiments is performed. Alternatively, the mass spectrometer output may be transmitted and/or received in any suitable way, for example, the output may be encoded and transferred from the mass spectrometer to a computer using a portable storage device, and embodiments of the invention are not limited in this respect.

The phraseology and terminology used herein is for the purpose of description and should not be regarded as limiting. As used herein, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

Additionally, the use of “including,” “comprising,” or “having,” “containing,” “involving,” and variations thereof herein, is meant to encompass the items listed thereafter and equivalents thereof as well as additional items.

What is claimed is:

1. A computer system for organizing output from a mass spectrometer, the output comprising a list of a plurality of detected proteins from a sample, each of the plurality of detected proteins having constituent peptides, each constituent peptide having at least one molecular property, the computer system comprising:
at least one processor programmed to:
assign each of the constituent peptides for each detected protein into one of a plurality of bins based at least in part on the at least one molecular property of the peptide; and
calculating peptide counts indicating a number of said peptides in each bin for each detected protein; and
a display for displaying the peptide counts as a plurality of cells in a data structure, the display presenting the counts in a table having columns arranged along a first axis and rows arranged along a second axis, the intersection of a column and a row defining a cell, and in each row displaying data pertaining to a specific detected protein and in each column displaying peptide counts for one of said plurality of bins;
wherein the at least one processor is further programmed to assign a first visual parameter value to each column of the data structure and a second visual parameter value to each cell based on its respective peptide count.

2. The computer system of claim 1, wherein the first visual parameter value is a hue and the second visual parameter value is a saturation.

3. The computer system of claim 1, wherein the first axis is horizontal and the second axis is vertical.

4. The computer system of claim 1, wherein the first axis is vertical and second axis is horizontal.

5. The computer system of claim 1, wherein the table further comprises the name of at least one detected protein.

6. The computer system of claim 1, wherein the table comprises a first set of columns for displaying counts for a first experiment and a second set of columns for displaying counts for a second experiment.

7. The computer system of claim 6, wherein the at least one processor is further programmed to assign to first selected cells in the first set of columns a first background color and assign to second selected cells in the second set of columns a second background color.

8. The computer system of claim 7, wherein the first selected cells and the second selected cells have a value of zero.

9. The computer system of claim 1, wherein the at least one molecular property is an isoelectric point.

10. The computer system of claim 1, wherein the at least one molecular property is a molecular weight.

11. The computer system of claim 1, wherein the at least one molecular property is a measure of hydrophobicity.

12. The computer system of claim 1, wherein the peptide counts for each protein comprise a total number of peptides observed for the protein.

13. The computer system claim 1, wherein the peptide counts for each protein comprise a number of unique peptides observed for the protein.

14. The computer system of claim 1, wherein the peptide counts for each protein relate to a percent coverage of peptides for the protein.

15. A computer system for arranging peptide count information from a mass spectrometer experiment, the computer system comprising a processor, the processor programmed to:
receive the peptide count information from a mass spectrometer;
create a data structure, the data structure comprising, for each protein for which a peptide count is to be displayed, a plurality of cells, the plurality of cells being arranged along a first axis; and
populate the data structure with the peptide count information by assigning to each cell the peptide count obtained over a predefined range of pH.

16-90. (canceled)
